(19) United States
(12) Patent Application Publication
(10) Pub. No.: US 2024/0342218 A1
(43) Pub. Date: Oct. 17, 2024

(54) NEURAL PROGENITOR CELLS AND THERAPEUTIC USES OF SAME

(71) Applicant: University Health Network, Toronto (CA)

(72) Inventors: Michael George FEHLINGS, Toronto (CA); Mohammad KHAZAEI, Toronto (CA); Christopher S. AHUJA, Ajax (CA)

(73) Assignee: University Health Network, Toronto, ON (CA)

(21) Appl. No.: 18/294,700

(22) PCT Filed: Aug. 3, 2022
(86) PCT No.: PCT/CA2022/051178

§ 371 Date: Feb. 2, 2024

Related U.S. Application Data
(60) Provisional application No. 63/229,140, filed on Aug. 4, 2021.

(51) Int. Cl. A61K 35/30; A61P 25/00; C12N 5/0797

(52) U.S. Cl. CPC A61K 35/30; A61P 25/00; C12N 5/0623; C12N 2501/119; C12N 2501/13; C12N 2501/155; C12N 2501/385; C12N 2506/45

(57) ABSTRACT

The present disclosure relates generally to neural progenitor cells and therapeutic uses thereof. More particularly, the present disclosure provides cervical spinal cord-specific neural progenitor cells (cerNPCs), methods of producing cerNPCs, pharmaceutical compositions comprising cerNPCs. and methods of treating neurological diseases or disorders with the cerNPCs.

### CROSS REFERENCE TO PRIOR APPLICATIONS

This application claims priority under the Paris Convention to United States Provisional Patent Application No. 63/229,140 filed on Aug. 4, 2021, which is incorporated herein by reference as if set forth in its entirety.

### FIELD OF THE DISCLOSURE

The present disclosure relates generally to neural progenitor cells and therapeutic uses thereof. More particularly, the present disclosure provides cervical spinal cord-specific neural progenitor cells (cerNPCs), methods of producing the cerNPCs, pharmaceutical compositions comprising the cerNPCs, and methods of treating neurological diseases or disorders with the cerNPCs.

### BACKGROUND OF THE DISCLOSURE

Transplantation of tripotent neural progenitor cells (NPCs) is a promising therapeutic strategy for traumatic spinal cord injury (SCI), however, the optimal temporal and spatial developmental stage for these cells remains to be determined.

NPC-derived neurons have the potential to integrate into endogenous neural networks and reestablish interrupted neuronal pathways. Despite recent progress, the level of graft-host integration and the degree of intra- and trans-segmental relay circuits regenerated by the transplanted neurons has been modest¹⁻³. This is partly due to suboptimal differentiation of transplanted NPCs in the injured cord microenvironment to non-neuronal cells^{4,5} and partly due to the difference in the identity of transplanted NPCs within the spinal cord niche. Native NPCs, along the entire rostrocaudal neural axis, possess a unique region-specific identity (e.g. forebrain, midbrain, cervical, thoracic, etc.)⁶ with unique growth, migration and integration during development and into adulthood. These cells have distinct neural differentiation in terms of channel composition, axonal projection pattern and neurotransmitter phenotype. These distinct characteristics allow proper integration during development and in adulthood. Endogenous NPCs that are taken from embryonic tissue possess region-specific identities (e.g. forebrain, midbrain, cervical, etc.).

There are several neuronal subtypes present in various brain and spinal cord regions, which relay region-specific functions such as fine motor control, cognition & memory, and even respiration. To date, almost all pluripotent cell-derived NPCs used for transplantation into the spinal cord have possessed this rostral identity (brain telencephalon identity). These cells terminally differentiate into neuronal cell types (e.g. cortical, subcortical, or deep nuclear neurons including excitatory pyramidal neurons, expressing Calbindin or CART, corticothalamic glutamatergic neurons. cholinergic neurons) which do not developmentally reside in the cervical spinal cord explaining the limited integration of the transplanted cells. Moreover, after SCI there is significant loss of ventral motor neurons and spinal interneurons (Renshaw cells, paragriseal and inhibitory interneurons⁷) which are likely required for a successful cell replacement approach. Evidence is now emerging that the limited integration of transplanted cells after SCI may be in part due to the graft's discordant regional identity⁵. In fact, recent studies using human induced pluripotent stem cell (hiPSC)-derived cells have demonstrated that neural cell derivatives must possess a regional identity that mimics the respective endogenous central nervous system (CNS) tissue in order to effectively engraft and regenerate⁵.

Spinal identity cells can generate functional V2a interneurons and motor neurons, whereas brain identity cells are inefficient at differentiating into V2a interneurons and motor neurons. A plethora of research has focused on differentiating stem cells into a V2a excitatory interneuron phenotype to optimize forelimb and hindlimb functional recovery following SCI⁸. This appears to be true as embryonic stem cells (ESCs) differentiated to express Chx10+V2a interneurons have been shown to enhance functional recovery post-SCI⁹. However, improved functional recovery using V2a differentiated stem cells does not equate to optimal functional recovery in all contexts of SCI.

There is a need for improved treatment options for patients with neurological diseases or disorders, including patients suffering from spinal cord injury, for whom transplantation of NPCs with a spinal cord identity may be particularly beneficial.

### SUMMARY OF THE DISCLOSURE

The inventors have invented cervical spinal cord-specific neural progenitor cells (cerNPCs), methods of producing the cerNPCs, pharmaceutical compositions comprising the cerNPCs, and methods of treating neurological diseases or disorders with the cerNPCs.

In a first aspect of the present disclosure, an engineered cervical spinal cord-specific neural progenitor cell (cerNPC) is provided. The cerNPC has an increased expression of one or more Hox genes and a decreased expression of one or more of Gbx2, Otx2 and FoxG1 relative to a non-specific neural progenitor cell (NPC) or a forebrain-specific neural progenitor cell (fbNPC).

In an embodiment of the engineered cerNPC provided herein, the cerNPC is capable of differentiating into neurons, astrocytes and/or oligodendrocytes.

In an embodiment of the engineered cerNPC provided herein, the cerNPC is derived from a non-specific NPC that expresses one or more detectable markers that are Sox2, Pax6, nestin and/or vimentin.

In an embodiment of the engineered cerNPC provided herein, the one or more Hox genes comprise one or more of HoxA3, HoxA4, HoxA5, HoxB3, HoxB5, HoxB6, HoxB9, HoxC4, HoxC5, HoxC6, HoxC9, HoxD4 and HoxD9.

In an embodiment of the engineered cerNPC provided herein, the one or more Hox genes comprise HoxA5 and/or HoxB6.

In an embodiment of the engineered cerNPC provided herein, the one or more Hox genes are expressed from an Emx2 promoter, a FoxG1 promoter, a Gbx2 promoter and/or an Otx2 promoter.

In an embodiment of the engineered cerNPC provided herein, the one or more Hox genes are expressed from an Emx2 promoter.

In an embodiment of the engineered cerNPC provided herein, the one or more Hox genes are expressed from an inducible promoter.

In an embodiment of the engineered cerNPC provided herein, the cerNPC is a human cerNPC.

In an embodiment of the engineered cerNPC provided herein, the non-specific NPC that expresses the one or more detectable markers that are Sox2, Pax6, nestin and/or vimentin is (a) derived from a stem or progenitor cell, or (b) reprogrammed from a differentiated cell.

In an embodiment of the engineered cerNPC provided herein, the stem or progenitor cell is derived from adult tissue, fetal tissue, or embryonic stem cells.

In an embodiment of the engineered cerNPC provided herein, the stem or progenitor cell is an induced pluripotent stem cell.

In an embodiment of the engineered cerNPC provided herein, synaptic connectivity of the cerNPC with endogenous neurons is increased relative to synaptic connectivity of the NPC or the fbNPCs with endogenous neurons.

In an embodiment of the engineered cerNPC provided herein, electrical conductance of the cerNPC across a site of spinal cord injury is increased relative to electrical conductance of the NPC or the fbNPCs across a site of spinal cord injury.

In a second aspect of the present disclosure, a pharmaceutical composition is provided. The pharmaceutical composition comprises the engineered cerNPC of the first aspect and a pharmaceutically acceptable carrier, diluent or excipient.

In an embodiment of the pharmaceutical composition provided herein, the pharmaceutically acceptable carrier is a xenogen-free culture medium or matrix.

In an embodiment of the pharmaceutical composition provided herein, the pharmaceutically acceptable carrier is cerebrospinal fluid or synthetic cerebrospinal fluid.

In a third aspect of the present disclosure, a method of treating a neurological disease or disorder in a subject is provided. The method comprises administering a therapeutically effective amount of the engineered cerNPC of the first aspect or the pharmaceutical composition of the second aspect to the subject.

In an embodiment of the method of treating a neurological disease or disorder in a subject provided herein, the administering comprises transplanting the engineered cerNPC to the brain or spinal cord of the subject.

In an embodiment of the method of treating a neurological disease or disorder in a subject provided herein, the neurological disease or disorder is Parkinson's disease, Alzheimer's disease, Huntington's disease, Amyotrophic lateral sclerosis, Friedreich's ataxia, Lewy body disease, spinal muscular atrophy, multiple system atrophy, dementia, schizophrenia, paralysis, multiple sclerosis, spinal cord injury, brain injury, stroke, cranial nerve disorders, peripheral sensory neuropathies, epilepsy, prion disorders, Creutzfeldt-Jakob disease, Alper's disease, cerebellar/spinocerebellar degeneration, Batten disease, corticobasal degeneration, Bell's palsy, Guillain-Barre syndrome, Pick's disease, or autism.

In an embodiment of the method of treating a neurological disease or disorder in a subject provided herein, the neurological disease or disorder is spinal cord injury.

In a fourth aspect of the present disclosure, a use of the engineered cerNPC of the first aspect or the pharmaceutical composition of the second aspect in the manufacture of a medicament for treating a neurological disease or disorder is provided.

In an embodiment of the fourth aspect provided herein, the neurological disease or disorder is Parkinson's disease, Alzheimer's disease, Huntington's disease, Amyotrophic lateral sclerosis, Friedreich's ataxia, Lewy body disease, spinal muscular atrophy, multiple system atrophy, dementia, schizophrenia, paralysis, multiple sclerosis, spinal cord injury, brain injury, stroke, cranial nerve disorders, peripheral sensory neuropathies, epilepsy, prion disorders, Creutzfeldt-Jakob disease, Alper's disease, cerebellar/spinocerebellar degeneration, Batten disease, corticobasal degeneration, Bell's palsy, Guillain-Barre syndrome, Pick's disease, or autism.

In an embodiment of the fourth aspect provided herein, the neurological disease or disorder is spinal cord injury.

In a fifth aspect of the present disclosure, a method of producing cerNPCs is provided. The method comprises:
(a) expressing one or more Hox genes in stem or progenitor cells;
(b) differentiating the stem or progenitor cells into non-specific neural progenitor cells (NPCs);
(c) culturing the non-specific NPCs for about 4 days in media supplemented with B27, N2, about 30-50 ng/ml of FGF2 or an agonist or synthetic analog thereof, and about 150-250 ng/ml of FGF8b or an agonist or synthetic analog thereof to generate posteriorized NPCs;
(d) culturing the posteriorized NPCs for about 4 days in media supplemented with B27, N2, about 0.075-0.125 UM of retinoic acid (RA) or an agonist or synthetic analog thereof, and optionally about 75-125 μg/ml of Wnt3a or an agonist or synthetic analog thereof to generate caudalized NPCs; and
(e) culturing the caudalized NPCs in media supplemented with B27, N2, about 7.5-12.5 ng/ml of FGF2 or an agonist or synthetic analog thereof, about 7.5-12.5 ng/ml of EGF or an agonist or synthetic analog thereof and about 0.75-1.25 UM of 740Y-P or an agonist or synthetic analog thereof to generate cerNPCs.

In an embodiment of the method of producing cerNPCs provided herein, the one or more Hox genes comprise one or more of HoxA3, HoxA4, HoxA5, HoxB3, HoxB5, HoxB6, HoxB9, HoxC4, HoxC5, HoxC6, HoxC9, HoxD4 and HoxD9.

In an embodiment of the method of producing cerNPCs provided herein, the one or more Hox genes comprise HoxA5 and/or HoxB6.

In an embodiment of the method of producing cerNPCs provided herein, the one or more Hox genes are expressed from an Emx2 promoter, a FoxG1 promoter, a Gbx2 promoter and/or an Otx2 promoter.

In an embodiment of the method of producing cerNPCs provided herein, the one or more Hox genes are expressed from an Emx2 promoter.

In an embodiment of the method of producing cerNPCs provided herein, the one or more Hox genes are expressed from an inducible promoter.

In an embodiment of the method of producing cerNPCs provided herein, the stem or progenitor cells are human cells.

In an embodiment of the method of producing cerNPCs provided herein, the stem or progenitor cells are derived from adult tissue, fetal tissue, or embryonic stem cells.

In an embodiment of the method of producing cerNPCs provided herein, the stem or progenitor cells are induced pluripotent stem cells.

In an embodiment of the method of producing cerNPCs provided herein, the non-specific NPCs express one or more detectable markers that are Sox2, Pax6, nestin and/or vimentin.

In an embodiment of the method of producing cerNPCs provided herein, step (b) comprises differentiating the stem or progenitor cells into the non-specific NPCs using embryoid body formation or dual SMAD inhibition.

In an embodiment of the method of producing cerNPCs provided herein, step (e) comprises culturing the caudalized NPCs for a period of about 3-10 passages to generate the cerNPCs.

In certain embodiments of the method of producing cerNPCs provided herein, in step (c), the FGF2 or the agonist or synthetic analog thereof is present at a concentration of about 40 ng/ml and the FGF8b or the agonist or synthetic analog thereof is present at a concentration of about 200 ng/ml; and/or in step (d), the RA or the agonist or synthetic analog thereof is present at a concentration of about 0.1 μM and the Wnt3a or the agonist or synthetic analog thereof is present at a concentration of about 100 μg/ml; and/or in step (e), the FGF2 or the agonist or synthetic analog thereof is present at a concentration of about 10 ng/ml, the EGF or the agonist or synthetic analog thereof is present at a concentration of about 10 ng/ml, and the 740Y-P or the agonist or synthetic analog thereof is present at a concentration of about 1 μM.

In an embodiment of the method of producing cerNPCs provided herein, the synthetic analog of RA is EC23.

In an embodiment of the method of producing cerNPCs provided herein, the culturing is performed on a substrate or matrix comprising poly-L-lysine, laminin, poly-L-lysine/laminin, fibronectin, vitronectin, collagen, Matrigel™, or Geltrex™.

In an embodiment of the method of producing cerNPCs provided herein, the culturing is performed in plates coated with poly-L-lysine/laminin.

In an embodiment of the method of producing cerNPCs provided herein, the cerNPCs are capable of differentiating into neurons, astrocytes and/or oligodendrocytes.

In an embodiment of the method of producing cerNPCs provided herein, the cerNPCs have an increased expression of one or more Hox genes and a decreased expression of one or more of Gbx2, Otx2 and FoxG1 relative to the non-specific NPC or a forebrain-specific neural progenitor cell (fbNPC).

In an embodiment of the method of producing cerNPCs provided herein, the method further comprises a step of formulating the cerNPCs with a pharmaceutically acceptable carrier, diluent or excipient.

In an embodiment of the method of producing cerNPCs wherein the method further comprises a step of formulating the cerNPCs with a pharmaceutically acceptable carrier, the pharmaceutically acceptable carrier is a xenogen-free culture medium or matrix.

In an embodiment of the method of producing cerNPCs wherein the method further comprises a step of formulating the cerNPCs with a pharmaceutically acceptable carrier, the pharmaceutically acceptable carrier is cerebrospinal fluid or synthetic cerebrospinal fluid.

### BRIEF DESCRIPTION OF THE DRAWINGS

In order that the subject matter of the present disclosure may be readily understood, embodiments are illustrated by way of the accompanying drawings.

FIG. 1: Gene expression analysis of NPCs derived from adult human tissues. Three lines from each adult derived fbNPCs (cortical 1 to 3) cerNPCs (cervical 1-3) and thoracic-NPCs (Thoracic 1-3) were analyzed.

FIG. 2: An embodiment of an approach used to develop the protocol for generating cerNPCs of the disclosure.

FIG. 3: A) Gene expression analysis demonstrated that the best combination of factors for patterning towards cerNPCs is treatment of fbNPCs with FGF8 for 4 days and then treatment with RA for additional 4 days. B) The steps towards generating unpatterned-NPCs (fbNPCs) from hiPSCs.

FIG. 4: The four conditions used for refinement of a protocol to generate stable cerNPCs of the disclosure.

FIG. 5: The gene expression signature of cells in conditions A-D were compared to ctrl (cerNPCs) after 1 or 5 passage (P5) after removal of FGF8/RA and culturing cells in regular N2/B27 media. The most stable condition was condition C.

FIG. 6: The hiPSC derived cervical NPCs that has been generated using a protocol of the disclosure (FGF8/RA+ Emx2::HoxB6) has been clustered to same category as cervical NPCs derived from fetal or adult human tissue.

FIG. 7: Steps towards generation of cervical NPCs. A) Cervical NPCs express the cervical spinal cord Hox genes as opposed to conventional cortical identity NPCs which express FoxG1, Emx2 and Otx2. B) A comparative gene expression analysis revealed that the expression of pluripotent cell markers (Oct4, Nanog) was decreased, whereas expression of neural cell markers (Sox2, Pax6 and Nestin) was increased in both cortical and cervical identify lines compared to the original parental hiPSCs. Cortical NPCs showed higher expression levels of transcription factors Otx2 and FoxG1, markers of anterior identity cells, as compared to cervical NPCs. Conversely, cervical NPCs expressed increased levels of Nkx2.2, Nkx6.1, HoxA4 and HoxA5 transcription factors.

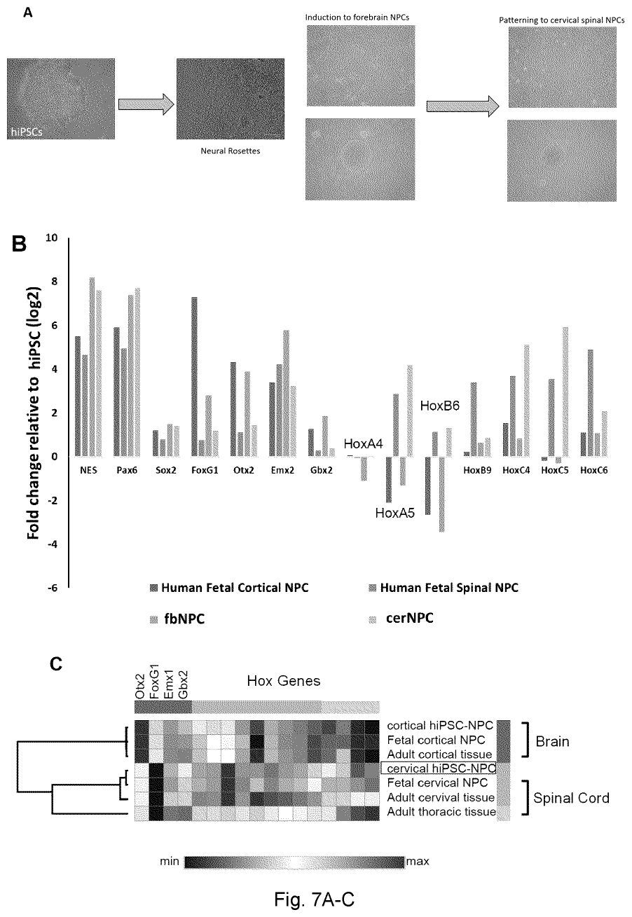

FIG. 8: Cervical spinal NPCs do not express brain Identity markers (FoxG1 and Otx2) in vitro, but express neural stem cell markers (Nestin and Pax6).

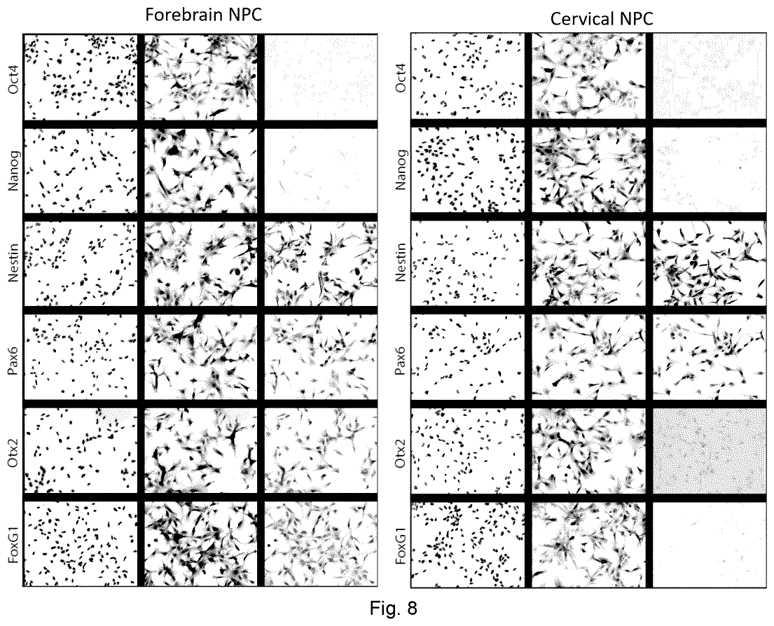

FIG. 9: Cervical spinal NPCs express cervical spinal cord specific marker HoxA4.

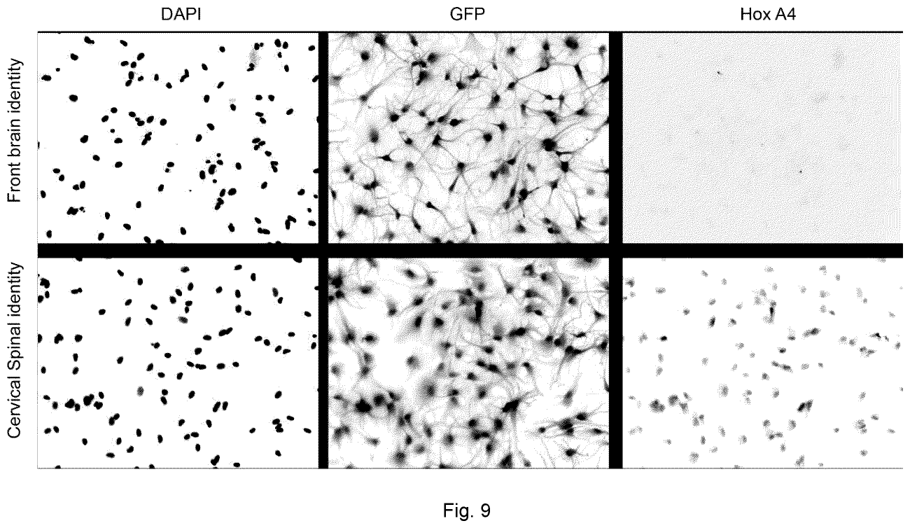

FIG. 10: In vitro differentiation assay of cerNPC lines. cerNPCs maintained their tripotency and the ability to generate neurons, oligodendrocytes and astrocytes in vitro.

FIG. 11: To investigate the in vivo behavior of cervical NPCs and compare them with cortical NPCs after transplantation, T-cell deficient RNU rats received a C6/7 cervical SCI followed by cell transplantation at 2 weeks post-injury. The results indicate that the cervical NPCs survived, migrated and differentiated in the injured spinal cord. Transplanted cells (GFP+) were found in both the white and gray matter. Grafted cervical NPCs were able to migrate as far as 15 mm rostral and caudal from the epicenter and predominantly migrated along white matter tracts, however, the migration of cortical NPCs was mainly limited to 9 mm.

FIG. 12: At 8 weeks post transplantation, both cortical and cervical NPCs differentiated into neurons, astrocytes, and oligodendrocytes in vivo, however, a proportion of cells in both lines remained in an undifferentiated Nestin positive state.

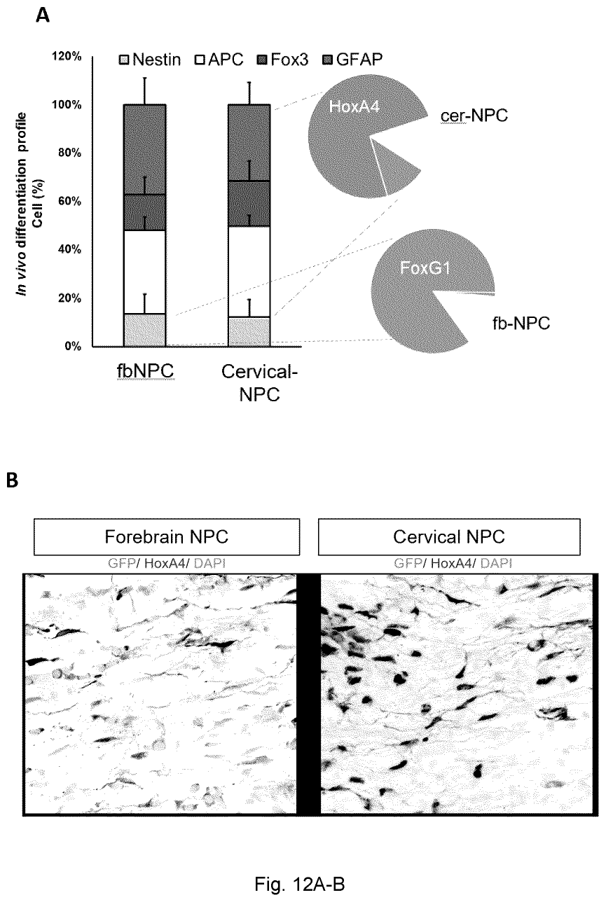

FIG. 13: Neurons differentiated from cervical NPCs expressed neuronal subtype specific transcription factors 12 weeks post transplantation, including Isl1, Hb9 (for motor neurons) FoxP1, Lhx1, Chx10 (for pre-motoneuron interneurons), Pax2 and Gata3 (for inhibitory inter-neurons). However, for cortical NPCs, motor neurons and subtypes of excitatory interneurons (FoxP1) were infrequently detected.

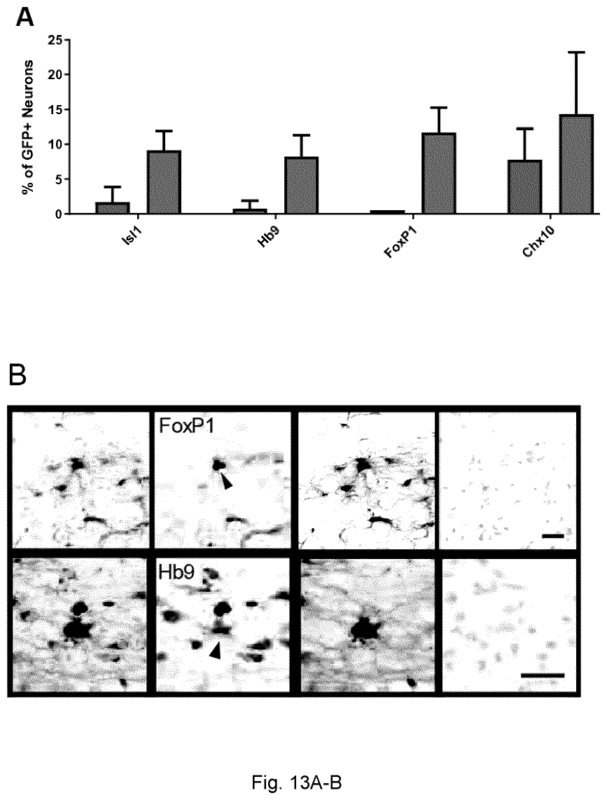

FIG. 14: Transplantation of cervical NPCs resulted in better functional recovery. Forelimb strength and trunk stability were assessed with grip strength and inclined plane behavioral tasks. Midterm Catwalk (Noldus Inc.) digital gait analysis shows significantly better forelimb stride length and swing speed recovery for cervical NPCs compared to cortical NPCs.

FIG. 15: Synapses can be identified by the apparent thickening of the apposed membranes of two cytoplasmic profiles. Connections between cervical NPCs and endogenous cells were identified. These new connections could potentially contribute to greater electrical transmission across the injury site. To test this, electrically-evoked compound action potential (CAP) transmission across the injury site (C5-T1) was analyzed. The CAP amplitude was significantly higher in the cervical NPCs transplant group compared to forebrain NPCs. To test the effect of cerNPC transplantation on electrical transmission, electrically-evoked compound action potential (CAP) transmission across the injury site (C5-T1) was analyzed. Traces of CAP amplitude were significantly higher in the cerNPCs transplant group compared to control fbNPCs.

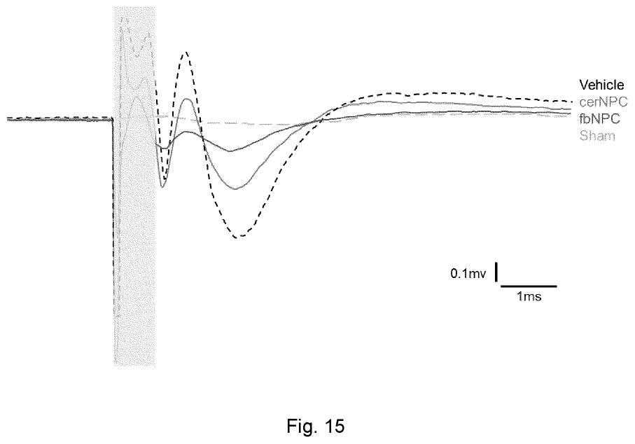

Other features and advantages of the present disclosure will become more apparent from the following detailed description and from the exemplary embodiments.

### DETAILED DESCRIPTION OF THE DISCLOSURE

### Definitions

The terminology used herein is for the purpose of describing particular embodiments only and is not intended to be limiting of example embodiments of the invention. Unless defined otherwise, all technical and scientific terms used herein generally have the same meaning as commonly understood by those of ordinary skill in the art. Generally, nomenclatures used in connection with neurology, immunology, transplant medicine, cell biology, molecular biology, cell culture, tissue culture, genetics, animal models of disease and injury, etc. described herein are those well-known and commonly used in the art. Nevertheless, definitions of selected terms are provided for clarity and consistency.

As used herein, the singular forms “a,” “an,” and “the,” are intended to include the plural forms as well, unless the context clearly indicates otherwise.

The phrase “and/or” should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, the phrase “one or more,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “one or more” refers, whether related or unrelated to those elements specifically identified. Thus, as a non-limiting example, “one or more of A and B” (or, equivalently, “one or more of A or B,” or, equivalently “one or more of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%.

When a range of values is listed herein, it is intended to encompass each value and sub-range within that range. For example, “1-5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4- 5 ng.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

The term “consisting of” and its derivatives, as used herein, are intended to be closed terms that specify the presence of stated features, integers, steps, operations, elements, and/or components, and exclude the presence or addition of one or more other features, integers, steps, operations, elements and/or components.

The term “isolated molecule” (where the molecule is, for example, a small molecule, a polypeptide, a polynucleotide, or an antibody or fragment thereof) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

The term “treatment,” “treat,” “treating” or “amelioration” as used herein is an approach for obtaining beneficial or desired clinical results. For purposes of this disclosure, beneficial or desired clinical results include, but are not limited to, one or more of the following: decreased extent of damage from a disease, condition, or disorder, decreased duration of a disease, condition, or disorder, reduction in the number, extent, or duration of symptoms related to a disease, condition, or disorder, an increase in the period of time prior to a relapse of a disease, condition, or disorder in a subject, and/or an increase in the disease-free or overall survival rate of a subject having a disease, condition, or disorder. The term includes the administration of the compounds, agents, drugs or pharmaceutical compositions of the present disclosure to prevent or delay the onset of one or more symptoms, complications, or biochemical indicia of a disease or condition; lessening or improving one or more symptoms; shortening or reduction in duration of a symptom; arresting or inhibiting further development of a disease, condition, or disorder; or decreasing the toxicity of a therapy. Treatment may be prophylactic (to prevent or delay the onset of a disease, condition, or disorder, or to prevent the manifestation of clinical or subclinical symptoms thereof) or therapeutic suppression or alleviation of symptoms after the manifestation of the disease, condition, or disorder. Treatment may also be maintenance therapy to decrease the chances that a disease, condition, or disorder will reoccur or to delay recurrence of a disease, condition, or disorder. The beneficial result may be an increase or decrease (as appropriate) of 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% relative to an appropriate control, for example, a subject that did not receive the therapy.

A “subject” is a vertebrate, preferably a mammal (e.g., a non-human mammal), and still more preferably a human. In an embodiment, the subject may be any human patient. In an embodiment, the subject may be limited to one or more patient subpopulations, such as, but not limited to, a female patient, a male patient, a geriatric patient, a pediatric patient, a patient with specific comorbidities and/or a patient with one or more genetic predispositions to a hereditary neurological disease or disorder.

The term “administering” or “administration” as used herein refers to the placement of cells, an agent, a drug, a compound, or a pharmaceutical composition as disclosed herein into a subject by a method or route which results in at least partial delivery of the composition to a desired site. The compounds and pharmaceutical compositions disclosed herein can be administered by any appropriate route which results in an effective treatment in the subject. Possible routes of administration of the compounds and pharmaceutical compositions disclosed herein include, but are not limited to, transplantation, implantation, intravenous, intraperitoneal, intramuscular, subcutaneous, transdermal, oral, buccal, sublingual, intranasal, or rectal routes of administration, or a combination thereof. The cerNPCs and other cell populations provided herein are administered, for example, by transplantation to the brain or spinal cord of a subject. The transplantation may be allogeneic transplantation or autologous transplantation. In some embodiments, the cerNPCs and other cell populations provided herein are transplanted to the site of a spinal cord injury or near the site of a spinal cord injury. In some embodiments, the cerNPCs and other cell populations provided herein are transplanted to the site of a brain injury or near the site of a brain injury.

The term “effective amount” or “therapeutically effective amount” as used herein is an amount sufficient to affect any one or more beneficial or desired results. In more specific aspects, an effective amount may alleviate or ameliorate one or more symptoms of a neurological disease or disorder, decrease the duration of time that one or more symptoms of a neurological disease or disorder are present in a subject, increase the period of time prior to a relapse of a neurological disease or disorder in a subject, and/or increase the disease-free or overall survival rate of a subject having a neurological disease or disorder. For prophylactic use, beneficial or desired results may include eliminating or reducing the risk, lessening the severity, or delaying the onset of a neurological disease or disorder or a particular stage/grade of the a neurological disease or disorder, including biochemical and/or histological symptoms of the a neurological disease or disorder, its complications and intermediate pathological phenotypes presenting during development of the a neurological disease or disorder. For therapeutic use, beneficial or desired results may include clinical results such as reducing one or more symptoms of a neurological disease or disorder; decreasing the dose or length of administration of other medications required to treat the neurological disease or disorder; enhancing the effect and/or reducing the toxicity of another medication; delaying the progression of the neurological disease or disorder a subject, decreasing the duration of time that one or more symptoms of neurological disease or disorder are present in a subject, and/or increasing the disease-free or overall survival rate of a subject having a neurological disease or disorder. An effective amount can be administered in one or more than one dose, round of administration, or course of treatment.

For purposes of this disclosure, an effective dosage of cells, a compound or a pharmaceutical composition is an amount sufficient to accomplish prophylactic or therapeutic treatment either directly or indirectly. As is understood in the clinical context, an effective dosage of cells, a compound, or a pharmaceutical composition may or may not be achieved in conjunction with another agent, drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved. The amount may vary from one subject to another and may depend upon one or more factors, such as, for example, subject gender, age, body weight, subject's health history, and/or the underlying cause of the disease, condition, or disorder to be prevented, inhibited and/or treated.

The term “pharmaceutically acceptable carrier, diluent, or excipient” as used herein includes any material which, when combined with an active ingredient, allows the ingredient to retain biological activity and is generally non-reactive with the immune system of a subject. Examples include, but are not limited to, any of the standard pharmaceutical carriers such as a phosphate buffered saline solution, water, emulsions such as oil/water emulsion, and various types of wetting agents. In some embodiments, diluents for aerosol or parenteral administration are phosphate buffered saline (PBS) or normal (0.9%) saline. In some embodiments, pharmaceutically acceptable carriers for transplantation of cells (e.g., cerNPCs) include, but are not limited to, xenogen-free culture media or matrixes, cerebrospinal fluid and synthetic cerebrospinal fluid. Compositions comprising such carriers are formulated by well-known conventional methods (see, for example, Remington's Pharmaceutical Sciences, 18th edition, A. Gennaro, ed., Mack Publishing Co., Easton, P A, 1990; and Remington, The Science and Practice of Pharmacy 20th Ed. Mack Publishing, 2000).

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Also included within the definition are polypeptides, oligopeptides, peptides and proteins having amino acid sequence identity to a given polypeptide, oligopeptide, peptide or protein. The percent identity can be, for example, at least about 50%, 55%, 60%, 65%, 70%, 75%, 80%, 85%, 90% 91%, 92%, 93%, 94%, 95%, 96%, 97%, 98%, 99%, or 100% amino acid identity to the given polypeptide, oligopeptide, peptide or protein. Also included within the definition are polypeptides, oligopeptides, peptides and proteins that have one or more conservative amino acid substitutions as compared to a given polypeptide, oligopeptide, peptide or protein. It is understood that the polypeptides can occur as single chains or associated chains. Methods for making polypeptides, oligopeptides, peptides and proteins are known in the art. In one embodiment the “polypeptide”, “oligopeptide”, “peptide” or “protein” is a mammalian protein, for example, a human protein.

As used herein, the term “stem cell” refers to a cell that can differentiate into more specialized cells and has the capacity for self-renewal. Stem cells may be totipotent (e.g., embryonic stem cells), pluripotent or multipotent. Stem cells may be derived from adult tissue, fetal tissue, or embryonic stem cells. Stem cells may be obtained from a patient (autologous stem cells) or a donor (allogeneic stem cells). Stem cells may be induced from a different cell type in vitro. In some embodiments, the stem cells are induced pluripotent stem cells (iPSCs). Methods for obtaining, deriving or producing stem cells are known in the art.

As used herein, the term “neural progenitor cell” or “NPC” refers to a neural lineage-committed cell that is capable of differentiating into one or more types of mature neural cells. “Tripotent” NPCs are capable of differentiating into three types of cells: neurons, astrocytes and oligodendrocytes. NPCs are also referred to in the art as, for example, “neural stem/progenitor cells” and “neural precursor cells”. The term “NPCs”, as used herein, encompasses neural progenitor cells, neural stem/progenitor cells, neural precursor cells, and other equivalent or similar terms used in the art. NPCs include non-specific NPCs, forebrain-specific NPCs (fbNPCs), midbrain-specific NPCs, cervical spinal cord-specific NPCs (cerNPCs), thoracic spinal cord-specific NPCs, etc. The terms “non-specific NPCs” and “unpatterned NPCs” are used interchangeably throughout the present disclosure. Non-specific NPCs can be derived from stem or progenitor cells using, for example, the embryoid body formation method or the dual SMAD inhibition method as disclosed herein.

As used herein, the phrase “expressing one or more Hox genes [in cells]” refers to a method step of genetically modifying, engineering or editing cells in order to overexpress the one or more Hox genes. Methods of genetic modification, engineering and editing are known to those skilled in the art, and various such methods may be used to overexpress the one or more Hox genes in accordance with the present invention. In some embodiments, the methods of genetic modification, engineering or editing include, but are not limited to, targeted insertion/integration of the one or more Hox genes using CRISPR/Cas9; random insertion/integration of the one or more Hox genes using retroviral vectors, lentiviral vectors or transposon systems (e.g., piggyBac); episomal expression of the one or more Hox genes from an episomal vector; and transient expression of the one or more Hox genes from a viral or non-viral transient expression vector. In some embodiments, the one or more Hox genes are expressed from an Emx2 promoter, a FoxG1 promoter, a Gbx2 promoter, an Otx2 promoter or an inducible promoter. In some embodiments, the one or more Hox genes are expressed from an Emx2 promoter. The one or more Hox genes include, but are not limited to, one or more of HoxA3, HoxA4, HoxA5, HoxB3, HoxB5, HoxB6, HoxB9, HoxC4, HoxC5, HoxC6, HoxC9, HoxD4 and HoxD9. In some embodiments, the one or more Hox genes comprise HoxA5 and/or HoxB6.

The nucleic acid sequences for genes disclosed herein and the amino acid sequences for proteins/polypeptides disclosed herein may be obtained from public databases. The accession numbers for a subset of the one or more Hox genes are provided in Table 1.

TABLE 1
Accession Numbers for a Subset of Hox Genes

| Gene | Entrez Gene ID Number | Description |
|---|---|---|
| HOXA3 | ENSG00000105997 | homeobox A3 HGNC: 5104 |
| HOXA4 | ENSG00000197576 | homeobox A4 HGNC: 5105 |
| HOXA5 | ENSG00000106004 | homeobox A5 HGNC: 5106 |
| HOXB3 | ENSG00000120093 | homeobox B3 HGNC: 5114 |
| HOXB5 | ENSG00000120075 | homeobox B5 HGNC: 5116 |
| HOXB6 | ENSG00000108511 | homeobox B6 HGNC: 5117 |
| HOXB9 | ENSG00000170689 | homeobox B9 HGNC: 5120 |
| HOXC4 | ENSG00000198353 | homeobox C4 HGNC: 5126 |
| HOXC5 | ENSG00000172789 | homeobox C5 HGNC: 5127 |
| HOXC6 | ENSG00000197757 | homeobox C6 HGNC: 5128 |
| HOXC9 | ENSG00000180806 | homeobox C9 HGNC: 5130 |
| HOXD4 | ENSG00000170166 | homeobox D4 HGNC: 5138 |
| HOXD9 | ENSG00000128709 | homeobox D9 HGNC: 5140 |

In the methods of producing cerNPC and other cell populations disclosed herein, the stem or progenitor cells from which the cerNPC and other cell populations are ultimately derived may be genetically modified, engineered or edited to express one or more Hox genes before differentiation of the stem or progenitor cells into non-specific NPCs. Alternately, the non-specific NPCs may be genetically modified, engineered or edited to express one or more Hox genes after differentiation from the stem or progenitor cells.

### General Techniques:

The practice of the present disclosure will employ, unless otherwise indicated, conventional techniques of chemistry, biochemistry, molecular biology, cell biology, cell and tissue culture, immunology, genetics, etc., which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Sambrook and Russell, Molecular Cloning: A Laboratory Manual, 3rd. ed., Cold Spring Harbor Laboratory Press, Cold Spring Harbor, NY (2001); Ausubel et al., Current Protocols in Molecular Biology, John Wiley & Sons, NY (2002); Harlow and Lane Using Antibodies: A Laboratory Manual, Cold Spring Harbor Laboratory Press, Cold Spring Harbor, NY (1998); Coligan et al., Short Protocols in Protein Science, John Wiley & Sons, NY (2003); Short Protocols in Molecular Biology (Wiley and Sons, 1999); and Immunobiology (C. A. Janeway and P. Travers, 1997).

Methods of Producing cerNPCs:
Step 1: Genetic Modification of Human Pluripotent Stem Cells (hPSCs) to Express HoxB6 Under Emx2 Promoter

This step can also be performed after the unpatterned NPCs are generated in Step 2.

For genetic modification, HoxB6 was expressed under Emx2 promoter. There are different methods for this genetic modification. The safest method that is compatible with downstream clinical application is insertion of Emx2::HoxB6 cassette in AAVS1 site using CRIPS/Cas9. The other method is to insert just HoxB6 ORF under the endogenous Emx2 promoter in a heterozygous manner.

Another method is integration of Emx2::HoxB6 cassette randomly into genome using retroviral, lentiviral transfection or transposon systems (like piggyBac).

Edited cells should be selected based on clonal culture method and each single clone is validated first using specific primes for transgenes and PCR and then whole genome sequencing.

The piggyBac transposon system was used to genetically modify the hPSC or NPCs derived from PSCs. piggyBac transposon systems has been widely used for non-viral gene integration and a promising candidate for applications of gene therapy in humans. The transgenes located between two inverted piggyBac elements are integrated into the target cell genome by transposase enzyme, but the rest of the vector which contains the enzyme is disintegrated after a couple of cell divisions and this will result in the establishment of stably transfected cells.

To transfect piggyBac-Emx2::HoxB6 vector into hiPSCs (or fbNPCs) the nucleofection method as described below was used. The stably transfected cells can be purified by applying puromycin, the gene for which is also integrated into the transfected cells.

### Procedure:

(1) Trypsinize hiPSC to single cells, neutralize the trypsin with DMEM supplemented with 10% FBS and count the live cells.
(2) Resuspend 1×106 cells in resuspension solution of Amaxa™ nucleofection kit (Cat #VPG-1004) for Mouse Neural Stem Cell which also works perfectly for human NPCs. Add 5 μg of piggyBac vector and electroporation cell in the cuvette using program A033.
(3) Transfer cells to a 6 cm Matrigel™ (or Geltrex™) (or PLL/Laminin in case of NPC) coated dish and add fresh culture media. For the first day in culture, supplement media with 10 μM ROCK inhibitor to increase the cell survival.
(4) Three to five days after transfection, add 2-10 μg/ml puromycin to culture media for 5-8 h and then change the media to fresh media. Generally using this treatment 90% of un-transfected cells dies whiten 24 h. Another round of purification can be done 3-5 days later to get a pure culture.

To transfect Using CRISPR/Ca9 for insertion of Emx2::HoxB6 cassette into AAVS1 site the nucleofection method as described below was used. The stably transfected cells can be purified by applying puromycine, which its gene is also integrated into the transfected cells.

### Procedure:

(1) Enzymatically detach hPSCs from culture surface using Accutase with Rock inhibitor, 10 μM. When cells are ˜90% confluent, each well of a 6-well plate will contain approximately 1.5 to 2×10\hat{ }6 cells.
(2) Perform nucleofection with CRISPR/Ca9 plasmids and the donor vector.
(3) After adding nucleofection solution, allow cuvettes to incubate for 10 min. This step increases the nucleofection efficiency, but must be limited to 10 minutes increases the risk of cell death.
(4) At 10 min, transfer cell suspension to hPSC Growth Media+ Rock inhibitor, 10 μM. For maximal recovery when nucleofecting, it is advisable to plate all 1×10\hat{ }6 cells onto a single well of an ECM coated plate.
(5) Incubate overnight in a 37° C. 5% CO2 humidified incubator.
(6) Exchange media with hPSC Growth Media and continue with normal hPSC culture protocols.
   Step 2: Generation of Unpatterned NPCs from hPSCs

Definitive human NPCs can be generated either with an Embryoid Body (EB) method (2a) or a dual-SMAD inhibition method (2b):

2a. Embryoid Body (EB) Method:

The embryoid body-formation technique is the older and more established method of unpatterned NPC generation from hPSCs 21. This method attempts to simulate the conditions of neurodevelopment to produce NPCs that more closely resembles the endogenous process, and is thus potentially less likely to result in aberrant modifications (genetic/epigenetic/signaling etc.) to resultant NPCs 21. Considering the neuroectodermal lineage is the default pathway for pluripotent cell differentiation, the embryoid body formation technique is a generally simple, robust and automatic procedure that requires few additional reagents beyond neuronal expansion media (NEM; see Table 3 for details) for solely deriving Sox1⁺ neural rosettes, and eventually nestin*, Sox2⁺, and Pax6⁺ NPC-containing neurospheres 21. Although the EB method is generally lengthier (˜40 days vs ˜23 for dual-SMAD inhibition), the overall yield of NPCs produced is generally higher 22. Since the EB formation technique involves merely the crude selection of neural rosettes based solely on shape and position at the NPC differentiation phase, there is a risk, however small, for carrying over undesired pluripotent cells into the next stage of culture^{22,23}.

Subsequently, NPCs are generated by the sequential arrangement of EBs and neuroepithelial-like rosettes. The EB-derived rosettes are then separated, gilded, and used as proliferative cells in the presence of fibroblast growth factor 2 (FGF2) and epidermal growth factor (EGF).

### Procedure 2a:

(1) To begin neural induction, separate hPSCs to single cells with Accutase®, then culture in suspension at the density of 1×10⁵ cells/mL on low adherent dishes in “Neural Expansion media (NEM)” (see Table 3)
Note: Starting with a homogeneous and healthy hPSC culture is important at this stage in order to achieve a higher efficiency of “pure” NPCs.
(2) The next day, replace half of the media with fresh media by tilting the plate. Such changes in half of the media are made daily for a period of five days. From day 5 onwards, cell aggregates, referred to as embryonic bodies (EB) form. The three-dimensional structure of these EBs generate a micro-tissue of embryonic structures, which acts to mimic the patterns observed within the naturally-occurring transition from embryonic-origin pluripotent cells to neural tube-like cells.
(3) On the 5th day of differentiation, EBs should be transferred to a 6 cm Matrigel-coated plate (Table 4) with NEM. After 24 hours, carefully examine cells under the microscope. EBs will have settled and be fully adherent to the plate.
(4) Change the media to fresh NEM on day 7. Change the media daily (using fresh NEM) until day 24.
(5) On day 24, the neuroepithelial cells in the center of the colonies form neural tube-like rosettes that are loosely attached. The first sign of the differentiation towards a neural lineage is the appearance of cells in the form of rosettes in the middle of the colonies, which occurs approximately 1 week after cultivation in NEM. The central, columnar cells in the rosettes, but not the cells in the periphery of each plate, will be positive for Pax6.
Note: neural rosettes at this stage are comprised of cells expressing early neuroectodermal markers such as Pax6 and Sox1 and are capable of differentiating into various region-specific neuronal and glial cell types in response to appropriate developmental cues.
(6) After two days, manually pick up the rosettes using fine pipette tips. The outer, non-neuronal cells should remain on the plate. Note that NPC purity is strongly dependent on proper manual selection of rosettes. Collect neuronal rosettes in 15 mL Falcon tubes in NEM (as many as necessary to accommodate the volume of rosettes), then triturate/thresh the rosettes to separate individual cells.
(7) The next step is to enrich NPCs by generating and isolating secondary rosettes. Resuspend the cells in NEM at a density of 1×10⁵ cells/cm² in the presence of Notch ligand DLL4 (500 ng/ml) and plate on poly-L-lysine (PLL)/laminin-coated plates (Table 4).
Note: The density of cells for re-plating at this stage is critically important for determining the differentiation state of cells. After isolation of rosettes they need to be re-plated in high density (1×10⁵ cells/cm²) in the presence of Notch ligand DLL4 to maintain their rosette structure. In contrast, culturing at low plating densities results in increased levels of unwanted differentiation and a significant reduction in rosette formation efficiency. DLL4 treatment increases the rosette structures, expression levels of NPC marker genes, and proliferation potential of NPCs 24. However, it should be noted that duration of DLL4 treatment is very important. Extended growth and passages at high cell densities and high DLL4 results in spontaneous differentiation and loss of rosette morphology.
Note: Treatment with DLL4 should not be extended for more than 1 passage. Temporal activation of Notch signaling is important for the transition from primitive to fully-definitive neural progenitor cell properties, and for maintenance of the definitive state. At this stage transient activation of Notch signaling maintains stem cells in an uncommitted state and promotes their self-renewal.

At this stage, the resultant culture now consists of pure populations of unpatterned NPCs that are positive for the expression of Nestin, Pax6 and Sox2 (but not Oct4)^{23,25}. After this step, it is possible to culture cells with one of two culture techniques: (a) suspension neurospheres or, (b) monolayer culture on substrate-coated plates.

By default, the hPSC-derived NPCs that are created using this method will have a dorsal anterior identity and be positive for the gene expression of orthodenticle homeobox 2 (OTX2), which encodes a homeodomain protein expressed in the fore and midbrain regions, yet be negative for the transcript for the homeobox protein, Hox-C4, an expression marker of spinal cord NPCs.

2b. Dual SMAD Inhibition Method:

Hereafter the “dual-SMAD inhibition” method utilized by Chambers et al. 2009, with a chemically defined culture of adherent cells²⁶ is elaborated on. The fundamental principal behind this method is, as the name describes, the concept of inhibiting the action of the intracellular signaling proteins, SMADs, thus disrupting two cell signaling and differentiation pathways: (1) the bone morphogenetic protein (BMP) pathways and, (2) the transforming growth factor beta (TGFβ) pathway, both of which act to inhibit the default pathway of neural induction^{25,26}.

The dual-SMAD inhibition method presents the advantage of mitigating the risk of non-neural cells remaining alongside NPCs in culture, which is unavoidable to some degree with the EB formation technique^{23,26}. Additionally, dual-SMAD inhibition is generally faster than EB formation, taking approximately 21-23 days to produce a sufficient culture of usable/expandable definitive NPCs, in comparison to the EB technique, which can take up to 40 days to reach an equivalent point^{23,25,26}. Despite these advantages, the final expected yield of NPCs produced will likely be lower with this method compared to the EB formation method²⁶.

### Procedure 2b:

(1) To begin neural induction, separate hPSCs to single cells with Accutase™, then culture the cells on Matrigel™ coated dishes at the seeding density of 25×10⁴ cells/cm² in “Neural Induction media (NIM)” (see Table 3). Supplement the media for initial seeding with 10 μM of Y-27632 (ROCK inhibitor).
Note: Starting with a homogeneous and healthy hPSC culture is important at this stage in order to achieve a higher efficiency of “pure” NPCs.
(2) Perform daily medium changes using fresh NIM supplemented with growth factors and morphogens, omitting the ROCK inhibitor (as it is no longer required after seeding cells).
(3) In about 8-10 days after cultivation in NIM, the first signs of differentiation towards a neuronal lineage will be apparent by the formation of column-shaped cells in the form of rosettes situated in the middle of the colonies. At this stage remove all dual SMAD inhibitors and switch media to NEM (Table 3).
   The next and final steps are similar to what is explained in steps 5-7 in Procedure 2a for the EB method, and are thus not repeated again here.

TABLE 2
List of Reagents

| | | Catalog |
|---|---|---|
| Reagent | Provider | Number |
| Purmorphamine | EMD Millipore | 540220 |
| Smoothened (Shh) | Millipore | 566660 |
| Agonist (SAG) | | |
| FGF | Wisent Inc. | 511-126-QU |
| EGF | Wisent Inc. | 511-110-EU |
| B-27 supplement, | Thermo Fisher Scientific | 12587010 |
| minus vitamin A | | |
| N2 supplement | Thermo Fisher Scientific | 17502048 |
| Heparin | Sigma | H3149 |
| DLL4 | Peprotech | 140-07 |
| DMEM:F12 | Thermo Fisher Scientific | 10565018 |
| Accutase | Thermo Fisher Scientific | A1110501 |
| Poly-L-Lysine | Sigma | A-005-C |
| Matrigel | Corning | 354230 |
| Laminin | Thermo Fisher Scientific | 23017015 |

TABLE 3
Culture Media Formulations

| | |
|---|---|
| Neural induction | DMEM/F12 medium, supplemented with sodium |
| medium (NIM) | pyruvate, glutamax, 1% penicillin, streptomycin |
| | solution, N2, B27 without vitamin A, 1% MEM |
| | (containing essential amino acids), FGF2 (20 |
| | ng/mL), EGF (20 ng/mL), heparin (1.25 U/L), |
| | TGFβ-inhibitor (SB 431542; 10 μM), |
| | BMP-inhibitor (LDN 193189; 100 nM) |
| NPC expansion | DMEM/F12 medium supplemented with sodium |
| medium (NEM) | pyruvate, Glutamax, 1% penicillin, streptomycin |
| | solution, N2, B27 without vitamin A, 1% MEM |
| | (containing essential amino acids), FGF2 (20 |
| | ng/mL), EGF (20 ng/mL) and heparin (1.25 U/L). |

TABLE 4
Coating Plates

| | |
|---|---|
| Matrigel | Thaw a 5 ml vial of Matrigel at 4° C. overnight. Matrigel |
| coating | should form a gel-like texture at room temperature (or |
| | any temperature above 10° C.). The next day, add cold |
| | (~4° C.) Matrigel to cold (~4° C.) culture medium to a |
| | final concentration of 3 mg/ml. Add 50 μl of diluted |
| | Matrigel to every 1 cm² of growth surface, so as to |
| | cover the entire surface of each plate. Warm up the |
| | plates for 1 h at 37° C., then aspirate the remaining |
| | coating solution. |
| PLL/laminin | Make a poly-L-lysine (PLL, molecular weight of 30,000 |
| coating | to 70,000 Daltons) solution of 0.1-1 mg/ml in 0.15 m |
| | borate buffer (pH 8.3), then filter sterilize with a |
| | 0.2 micron filter. Add enough solution to cover the |
| | surface of each plate, then incubate at room temperature |
| | for 2 hours. Aspirate the solution then add the laminin |
| | solution (10 μg/ml) to the surface and incubate for 1 |
| | hour at 37° C. Aspirate the solution and wash gently |
| | with 1 × PBS. |

### Step 3: Patterning NPCs Towards a Cervical Spinal Cord-Specific Identity

To generate cerNPCs, cells were patterned using a stepwise treatment of morphogens²⁷. Patterning of unpatterned NPCs (fbNPCs) towards a cervical spinal cord identity is modelled on the developmental cues that are involved in the formation of the spinal cord during embryogenesis.

Early in differentiation, all neural cells have a rostral identity. However, future caudal cells gradually become posteriorized and then obtain characteristics of the caudal midbrain and spinal cord. FGFs, Wnt, retinoic acid (RA), and Shh are involved in this spinal cord specification and subsequent elongation. The concentration and timing of these factors is critical. Among RA, Wnt, and FGF signals, RA causes the strongest level of caudalization: inducing suppression of forebrain differentiation and the promotion of caudal CNS specification.

The region of the neural plate giving rise to the spinal cord is specified in an FGF-dependent manner. Several FGFs, including FGF3, FGF4, FGF8, FGF13, FGF18, are involved in spinal cord specification. In vitro experiments have shown that exposure of the neural tissue to increasing FGF levels results in progressively elevated levels of HOXC6, HOXC8, HOXC9, or HOXC10^{28,29}. Furthermore, several signaling pathways influence FGF8 expression. The Wnt and Shh pathways, which are active in the caudal region of the neural tube, can themselves increase FGF8 levels 30.31

### Procedure:

(1) Dissociate cells into single cells using Accutase. Plate cells at 1×10⁴ cells/cm² on PLL/laminin pre-coated standard culture plates in DMEM: F12 media supplemented with B27, N2, FGF2 (40 ng/ml), and FGF8b (200 ng/ml). Incubate under standard conditions for four (4) days²⁷.
Note: In this step 2× more FGF2 and a high concentration of FGF8 are used. In the embryo, caudal cells are exposed to select FGFs for longer periods of time than rostral cells they are involved in regionalization of the spinal cord along the rostral-caudal axis. During later stages of spinal cord elongation, FGF8 is expressed more broadly. Expression of FGF8 continues for several days but declines toward the final stages of somitogenesis and the cessation of axis elongation^{32,33}. Treatment with FGF8 at this concentration and time period results in posteriorization of the cells. The posteriorized NPCs produced at the end of this stage express more Hox genes, such as HoxA4, and have reduced expression of brain markers such as Gbx2, Otx2 and FoxG1 compared to un-patterned cells. Posteriorized NPCs are equally tripotent with the same differentiation profile as un-patterned NPCs. The ability to form neurospheres and the proliferation rate of posteriorized NPCs are marginally higher than un-patterned NPCs.
(2) On day 4, use Accutase to passage cells to new PLL/laminin pre-coated standard culture plates in DMEM: F12 media supplemented with B27, N2, 0.1 μM EC23, [and optionally Wnt3a (100 μg/ml)]. Incubate for an additional 4 days.
Note: In this step caudalization of cells using RA or the synthetic retinoid analogue, EC23 is induced. Using EC23 is preferred as it is more photostable at incubation temperatures. The distribution of RA in an embryo induces positional specification of neural stem cells during development. RA promotes the caudalization of cells in a concentration-dependent manner^{34,35}.
   FGF and RA signaling are not sufficient (alone or together) to induce caudal characteristics in neural cells grown in vitro, thus Wnt signaling (Wnt3a) is further enhance the specification of neural cells to a caudal identity³⁶.
(3) On day 8, use Accutase to passage cells to new PLL/laminin pre-coated standard culture plates in DMEM: F12 media supplemented with B27, N2, FGF2 (10 ng/ml), EGF (10 ng/ml) and 740Y-P (1 μM).
Note: Treatment with RA [and optionally Wnt] for 4 days results in caudalization of cells. These caudalized NPCs express Hox genes such as HoxA4. RA or EC23 stabilize the caudal identity of the NPCs. This RA pathway activation results in a significant reduction (to nearly no expression) of Gbx2, Otx2 and FoxG1 levels compared to unpatterned cells NPCs or fbNPC. Cervical NPCs are also tripotent with the same differentiation profile as fbNPCs. However, the ability of cervicalNPCs to form neurospheres and their proliferation rate are significantly reduced compared to un-patterned NPCs.
(4) Passage cervical-NPCs for 3-10 passages in DMEM: F12 supplemented with B27, N2, FGF2 (10 ng/ml), EGF (10 ng/ml) and 740Y-P (1 M) until the identity of cells is cervical NPCs can be passed and maintained in this media for up 3-5 more passages.
Note: During the maintenance period, the proliferation rate of cells is reduced. To generate sufficient numbers of cells, prolonged culture for several passages is required. The concentration of FGF2 cannot be increased at this stage. To overcome this problem, 740Y-P is added which is as effective as FGF2 at promoting neuronal cell survival and proliferation via the PI 3-kinase-Akt pathway³⁷. The effect of 740Y-P is dose dependent.
   It is preferable to use cerNPCs between about passages 3-10 (about P3-P10). However, later passage cells may develop NPCs with mixed identity and cells that generate more GABA-ergic interneurons.
   After each passage, add 10 M Rock inhibitor (Y-27632) on day 1 of culture.

### Methods of Treating a Neurological Disease or Disorder:

The cerNPCs and other cell populations provided herein may be used for treating neurological diseases or disorders. In an embodiment, a population of cerNPCs is produced according to the methods disclosed herein; optionally formulated with a pharmaceutically acceptable carrier, diluent or excipient; and administered to a subject suffering from a neurological diseases or disorder. The neurological disease or disorder may be Parkinson's disease, Alzheimer's disease, Huntington's disease, Amyotrophic lateral sclerosis, Friedreich's ataxia, Lewy body disease, spinal muscular atrophy, multiple system atrophy, dementia, schizophrenia, paralysis, multiple sclerosis, spinal cord injury, brain injury, stroke, cranial nerve disorders, peripheral sensory neuropathies, epilepsy, prion disorders, Creutzfeldt-Jakob disease, Alper's disease, cerebellar/spinocerebellar degeneration, Batten disease, corticobasal degeneration, Bell's palsy, Guillain-Barre syndrome, Pick's disease, or autism.

In some embodiments, the neurological disease or disorder is a traumatic spinal cord injury or brain injury. In an embodiment, the neurological disease or disorder is a spinal cord injury.

The cerNPCs and pharmaceutical compositions provided herein may be administered to a subject in an effective amount or a therapeutically effective amount. A person of skill in the art would be able to determine such amounts based on such factors as the subject's size (e.g., weight), age and/or sex; the severity of the subject's symptoms; and the particular composition or route of administration selected. A person of skill in the art would also know how to select the proper route of administration and how to administer the cerNPCs or the pharmaceutical compositions provided herein to the subject. For example, the cerNPCs and other cell populations provided herein may be administered by transplantation to the brain or spinal cord of the subject. The transplantation may be allogeneic transplantation or autologous transplantation. In some embodiments, the cerNPCs or other cell populations provided herein are transplanted to the site of a spinal cord injury or near the site of a spinal cord injury. In some embodiments, the cerNPCs or other cell populations provided herein are transplanted to the site of a brain injury or near the site of a brain injury.

The dosage of the cerNPCs or the pharmaceutical compositions provided herein varies depending on many factors, such as pharmacodynamic properties, mode of administration, the age, health and weight of the recipient, the nature and extent of the symptoms, the frequency of the treatment and the type of concurrent treatment, if any, and the clearance rate. One of skill in the art can determine the appropriate dosage based on the above factors. In some embodiments, the cerNPCs or the pharmaceutical compositions are administered initially in a suitable dosage that is adjusted as required, depending on the clinical response. As a representative example, an appropriate dosage of cerNPCs for transplantation into the brain or spinal cord of a subject is in the range from about 10⁵ cells to about 10¹⁰ cells, or from about 10⁶ cells to about 10⁹ cells, or from about 10⁷ cells to about 10⁹ cells, or from about 10⁸ cells to about 10⁹ cells. In some embodiments, an appropriate dosage of human cerNPCs for transplantation into or near the site of a spinal cord injury of a subject is in the range from about 1×10⁸ cells to about 4×10⁸ cells.

### EXAMPLES

The disclosure is further described by reference to the following examples. These examples are provided for purposes of illustration only and are not intended to be limiting unless otherwise specified. Thus, the disclosure should in no way be construed as being limited to the following examples, but rather, should be construed to encompass any and all variations which become evident as a result of the teaching provided herein.

### Example 1: Materials and Methods

### Animal Care and Use:

All animal procedures were carried out in accordance with the University Health Network (UHN) Animal Care facility (Toronto, ON) and in accordance with the policies established by the Canadian Council of Animal Care's guide to the care and use of experimental animals.

### Rat Model of Cervical Spinal Cord Injury:

Animals were subject to a contusion cervical SCI using a modified aneurysm clip, which has been extensively characterized and previously described¹³. Briefly, adult female athymic RNU rats (NIH-Foxn1^{mu}) rats (180-200 g; Charles River, Montreal, Canada) were deeply anesthetized using 4% isoflurane, and were sedated for the remainder of the surgery under 2% isoflurane. Animals received a two-level laminectomy of cervical vertebral segments C6-C7. A modified clip calibrated to a closing force of 21.5 g was applied extradurally to the cord for 1 minute and then removed. Sham-operated rats received the laminectomy without the clip compression injury. A small square of Surgifoam (Ethicon Endo-Surgery, Inc., Cincinnati, OH) was placed over the injury site, and the overlaying muscle and skin were sutured. Postoperatively, animals were treated with analgesics (0.05 mg/kg buprenorphine and 5 ml saline) and saline (0.9%; 5 mL) to prevent dehydration. Animals were housed individually in standard rat cages with absorbent bedding at a temperature of 27° C. for recovery. Injured animals had their bladders manually expressed three times daily until natural bladder function returned.

gRT-PCR:

Quantitative RT-PCR (qRT-PCR) was used to examine the expression profile of pluripotency or differentiation markers in cells. For characterization NPCs, neural, astrocytic and oligodendroglial markers (see Table 6 for list of primers) were examined with the use of appropriate primers. mRNA was isolated using the RNAeasy mini kit (Qiagen, Hilden, Germany). A Nanodrop spectrophotometer was used to evaluate the concentration and purity of the mRNA. cDNA was synthesized using SuperScript® VILO cDNA Synthesis Kit (Life Technologies, Carlsbad, CA) with random hexamere primers according to manufacturer instructions. RT-PCR was performed using TAQman design primers with FAST taqman master mix under recommended thermocycling parameters on a 7900HT Real time PCR system. Samples were run in triplicate. Values were normalized to the GAPDH housekeeping gene. For examination of the neural progenitor, neuronal, astrocytic and oligodendroglial markers, results were normalized to GAPDH and to hiPSC source. Gene expression level were compared using the 2-1\CT method.

### Neurosphere Assay:

hiPSC-NPCs were plated at clonal density of 10 cells/μL in uncoated 24-well plates (Nunc, Rochester, NY). Neurospheres>50 μm in diameter were quantified after 7 days of undisturbed culture. Just prior to imaging, the content of each well was transferred to Matrigel coated dish, incubated for 30 min and fixed with 4% PFA. To assess long-term self-renewal, primary neurospheres were passaged for 3 times. For each passage, each neurosphere was transferred to a 500-μl tube containing 200 μl NEM, and then triturated and plated at clonal density in a final volume of 500 μl medium into a new 24-well plate.

### In Vitro Differentiation and Immunocytochemistry:

In order to examine the differentiation potential of the hiPSC-NPCs and analyze whether there were any differences between Cervical NPCs vs. forebrain NPCs, cells were differentiated in vitro by culturing them in pro-neurogenic, -oligodendrogenic and—astrocytogenic conditions. For the neurogenic condition, cells were cultured for two weeks on PLL/laminin substrate in a neurobasal medium supplemented with B27, N2, Retinoic Acid (0.1 UM), cAMP (100 ng/ml), and brain-derived neurotrophic factor (BDNF, 10 ng/ml; PeproTech, Rocky Hill, NJ) in the absence of FGF and EGF. To induce astrocyte differentiation, hiPSC-NPCs were cultured on Matrigel in DMEM/F12 supplemented with B27, 0.1% fetal bovine serum (FBS), BMP4 (10 ng/ml, Peprotech) and CNTF (5 ng/ml; PeproTech) for 14 days. To promote oligodendrocyte differentiation, hiPSC-NPCs were cultured on Matrigel in DMEM supplemented with N2 supplement, and treated for 3 days with Retinoic Acid (0.1 μM). The Shh agonist, Purmorphamine (1 μM) was added from day 2 for 7 days. On day 7, PDGF-AA (20 ng/ml) was added for another 7 days. To enhance the maturation of oligodendrocytes, triiodothyronine (T3) (30 ng/ml; Sigma-Aldrich) was added during the final phase of differentiation for 6 days. Morphological analyses and immunostaining with markers for neurons and astrocytes were performed after 14 days in vitro differentiation and after 21 days for oligodendrocytes.

### Cell Proliferation Assay:

NPCs were plated on laminin coated 96-well tissue culture plates (removable strip plates, Corning) at the density of 1×10³ cells/100 μl/well and the cell number was determined at 12 h, 24 h, 48 h and 72 h, after plating using a BrdU cell proliferation assay (abcam #ab126556) as recommended by the manufacturer.

### Intraspinal Cell Transplantation:

Rats received NPC transplantation 2 weeks (subacute) following SCI. hiPSC-NPCs were dissociated into a single-cell suspension by using Accutase at a concentration of 5×10⁴ cells/μl in neural expansion medium and were transplanted (2 μl) bilaterally at 4 positions 3-5 mm caudal and rostral to the lesion epicenter bilateral to the midline. Injections sites were situated approximately 2 mm from the midline and entered 1 mm deep into the cord.

### Tissue Collection and Histology:

Eight weeks after transplantation, rats were anesthetized and transcardially perfused with PBS and fixed with 4% PFA. Tissues were collected and post-fixed for 6 h in 4% PFA and transferred into 30% sucrose for 24 hours prior to sectioning (30 μm) on a cryostat (CM3050 Leica).

### Lesion Site Analysis:

Tissue sparing was analyzed 10 weeks after SCI, at the center of the lesion, 2400 μm rostral and 2400 μm caudal to the injury epicenter. Sections were stained with the myelin-selective stain luxol fast blue (LFB) and hematoxylin and eosin (H&E) which stains all the cell nuclei and cytoplasmic proteins. A blinded investigator performed LFB and H&E analyses on tissue. Total extent of injury was ±1440 μm. Unbiased measurements were made with a Cavalieri volume probe using Stereo Investigator (MBF Bioscience, Williston, VT) for total area, gray matter, white matter, cavitation, and total lesion reported as volumes. Cystic cavity was defined as any region within the total circumference that was devoid of tissue. Lesional tissue included any abnormal-appearing tissue such as demyelinated white matter, fibrous and glial scarring with the following aberrant histology; small round cysts, irregularly shaped vacuoles, disorganization of both white and gray matter and eosinophilic neurons. Calculations and analyses were done for tissue sections every 240 μm¹⁵.

### Immunohistochemical Staining:

Frozen slides were removed from the −80° C. freezer and tissue sections, at similar distances from the lesion epicentre, were allowed to dry in an indirect breeze for 15 min and outlined with a PAP pen. After blocking in blocking buffer (2.5% goat serum, 5% nonfat milk, and 0.3% Triton X-100) the samples were incubated with primary antibodies overnight at 4° C. The following day, secondary antibodies were applied (Alexa fluor 488, 568 or 647, 1:1000, Molecular Probes) at room temperature for 1 h. Slides were cover-slipped with Mowiol containing DAPI.

### Microscopy/Stereoinvestigator:

Cell quantification in the spinal cord tissue of transplanted rats was performed in an unbiased manner using StereoInvestigator optical fractionator software (MBF Bioscience, Williston, VT) on a Nikon Eclipse E800 microscope using 20× magnification. For quantification of cell survival, the total number of GFP-expressing/DAPI-positive cells were counted over a distance of 10 mm inclusive of the injury epicenter. Every sixteenth section was counted (480 μm apart) to avoid repeated counting of the same cells. Only the GFP cell bodies that contained a nucleus stained with DAPI were quantified to avoid counting nonviable GFPs or other autofluorescent objects. Confocal images were captured on a Zeiss LSM 510 laser confocal microscope.

### Behavioral Tests:

During the 8 week post-trasnplantation period, rats from each experimental group underwent weekly neurobehavioural testing to assess forelimb strength, digital dexterity, trunk stability and function. Two independent, blinded observers measured each parameter. Forelimbs were assessed using a force meter for grip strength¹⁶. Trunk and forelimb strength were also assessed using the inclined plane test¹⁷ in which rats were placed on an inclined plane platform, while the angle was gradually increased until rats were unable to hold their position for 5 seconds. The last angle that rats were able to hold for 5 seconds was recorded. Forelimb gait analysis was performed using the Catwalk gait assessment system (Noldus Information Technology, Wageningen, Netherlands)^{18,19}. All animals underwent open field motor scoring using the Basso, Beattie, and Bresnahan (BBB) scale weekly²⁰. To assess the effect of transplanted cells on thermal allodynia, the tail flick test was performed. Animals were wrapped in a soft towel to settle them. The dorsal surface of the tail between 4 and 6 cm from the tip was exposed to a beam of light calibrated to 50° C. generated from an automated machine (IITC Life Science, Woodland Hills, CA). The timer was stopped when the animal flicked its tail away, indicating an aversive response. Latency was measured at 15-minute intervals over 3 consecutive trials, with mean latency reported. If animals did not respond to the beam by 20 seconds, the procedure was stopped, and latency was scored as 20.

### Immuno-Electron Microscopy:

Frozen sections were incubated with anti-GFP mouse monoclonal antibody, and then incubated with nanogold-conjugated anti-mouse IgG secondary antibody (1:100 Invitrogen). Sections were fixed with 2.5% Glutaraldehyde. HQ-Silver kit was used to enhance the gold signal (Nanoprobes), and the sections were post-fixed with 0.5% OsO4, dehydrated through graded ethanol, and embedded into Epon. After complete polymerization at 60 degree for 72 hours, ultrathin sections (70 nm thick) were prepared, and stained with uranyl acetate and lead citrate, and observed under a transmission electron microscope (TEM, JEOL 1400plus).

### Patch Clamp for Voltage-Gated Currents and Action Potentials Recording:

Voltage-gated currents and action potentials (APs) were recorded using whole-cell patch clamp method in cultured neurons derived from GDNF-hiPSC-NPCs and GFP-hiPSC-NPCs. Patch pipettes resistance was 3-5 MΩ when filled with an intracellular solution (20 mM KCl; 121 mM K-gluconate; 0.5 mM CaCl₂; 1 mM MgCl₂; 10 mM EGTA; 10 mM HEPES and 4 mM MgATP; pH 7.3 the osmolality of 290-300 mOsm). Extracellular solution consisted of 145 mM NaCl, 2.5 mM KCl, 2 mM CaCl₂), 1 mM MgCl₂, 10 mM HEPES, 10 mM Glucose, pH 7.4 and the osmolality of 300-320 mOsm. Undergone sodium currents recording, membrane potential was held at −80 mV and depolarized to +30 mV with an increment of 10 mV step.

For the measurement of Aps, membrane potential was recorded at −60˜−70 mV and 10 pA stepped currents were injected into recorded neurons. Data were acquired using a multiClamp 700A and pClamp 10 software interfaced to a Digidata 1322A acquisition board (Molecular Devices, CA,) and signals were filtered at 10 KHz and digitized at 250 KHz.

### Statistical Analyses:

All animals were randomized into injury and treatment. All data were collected and quantified in a blinded fashion. Results are stated in mean±standard error of the mean (SEM). Gene expression and immunohistological data were analyzed using Student's t tests. Histomorphometric and behavioral data were analyzed using two-way analysis of variance (ANOVA) with Tukey's post hoc test. The significance level for all analyses was set at ** p<0.01 or *p<0.05. Data were analyzed with Prism 6 (GraphPad Software).

TABLE 5
Reagents and Resources

| | | |
|---|---|---|
| REAGENT | | |
| or RESOURCE | SOURCE | IDENTIFIER |

Antibodies

| | | |
|---|---|---|
| GFAP | EMD Millipore | MAB3402 |
| O1 | EMD Millipore | MAB344 |
| βIII-Tubulin | EMD Millipore | MAB1637 |
| APC-CC1 | EMD Millipore | OP80 |
| Fox3 | Biolegend | SIG-39860 |
| Nestin | EMD Millipore | ABD69 |
| Caspase 3 | Cell Signaling | 9665 |
| Olig2 | EMD Millipore | MABN50 |
| GDNF | Abcam | ab18956 |
| GFR1α | Abcam | ab8026 |
| GAPDH | EMD Millipore | MAB379 |
| RET | Cell Signaling | 3220 |
| p-RET | Cell Signaling | 3221 |
| Chat | EMD Millipore | AB143 |
| β-actin | Novus Biologicals | AC-15 |
| Ki67 | Novus Biologicals | NB600-1209 |
| Oct4 | Stemgent | 09-0023 |
| Nanog | Peprotech | 500-P236 |
| Pax6 | EMD Millipore | MAB5554 |

Chemicals, Peptides, and Recombinant Proteins

| | | |
|---|---|---|
| BDNF | Peprotech | 450-02 |
| GDNF | Peprotech | 450-10 |
| CNTF | Peprotech | 450-13 |
| Purmorphamine | EMD Millipore | 540220 |
| HGF | Peprotech | 100-93H |
| FGF | Winsent Inc. | 511-126-QU |
| EGF | Winsent Inc. | 511-110-EU |
| B-27 supplement, | Thermo Fisher Scientific | 12587010 |
| minus vitamin A | | |
| N2 supplement | Thermo Fisher Scientific | 17502048 |
| Heparin | Sigma | H3149 |
| mTeSR1 | STEMCELL Technologies | 05850 |
| DMEM:F12 | Thermo Fisher Scientific | 10565018 |
| Accutase | Thermo Fisher Scientific | A1110501 |
| Lipofectamine 3000 | Thermo Fisher Scientific | L3000008 |
| Matrigel | Corning | 354230 |
| Laminin | Thermo Fisher Scientific | 23017015 |

Critical Commercial Assays

| | | |
|---|---|---|
| IL-6 Quantikine | R&D systems | R6000B |
| ELISA Kit | | |
| Human Growth Factor | Abcam | ab134002 |
| Antibody Array | | |
| BMP-2 Quantikine | R&D systems | DBP200 |
| ELISA Kit | | |
| TGF-beta 1 Quantikine | R&D systems | MB100B |
| ELISA Kit | | |
| TiterTACS In Situ | R&D systems | 4822-96-K |
| Colorimetric TUNEL Kit | | |
| GDNF Emax | Promega | G7620 |
| ImmunoAssay Systems | | |
| BrdU Cell Proliferation | Abcam | ab126556 |
| ELISA Kit | | |

Experimental Models: Cell Lines

| | | |
|---|---|---|
| Human: induced | NIH Center for | BC1 |
| pluripotent stem | Regenerative Medicine | |
| cell BC1 | | |
| Human: iPSC-derived | This application | N/A |
| NPCs | | |

Experimental Models: Organisms/Strains

| | | |
|---|---|---|
| Rat: NIH-Foxn1^{mu} | Charles River | N/A |

Recombinant DNA

| | | |
|---|---|---|
| Quantitative real- | See Table 6 below | N/A |
| time PCR Primers | | |

Software and Algorithms

| | | |
|---|---|---|
| Catwalk | Noldus | 10.6 |
| ImageJ | NIH | 1.6.0_24 |
| pCLAMP | Molecular Devices | 10 |
| Stereo Investigator | MBF Bioscience | 10 |
| GraphPad Prism 6 | GraphPad | 6 |

TABLE 6
List of TaqMan ™ qPCR probes (from Applied Biosystems)

| Gene Symbol | Probe ID | Gene Symbol | Probe ID |
|---|---|---|---|
| ACKR3 | Hs00664172_s1 | FOXO4 | Hs00936217_g1 |
| ACTA2 | Hs00426835_g1 | GABRB3 | Hs00241459_m1 |
| ACTA2 | Hs00909449_m1 | GAPDH | Hs02758991_g1 |
| ACTB | Hs01060665_g1 | GAPDH | Hs02786624_g1 |
| ACTB | Hs99999903_m1 | GAPDH | Hs99999905_m1 |
| ALDH1L1 | Hs00201836_m1 | GBX2 | Hs00230965_m1 |
| ALDH1L1 | Hs01011071_m1 | GDF3 | Hs00220998_m1 |
| AQP4 | Hs00242342_m1 | GFAP | Hs00909233_m1 |
| ASCL1 | Hs04187546_g1 | GFAP | Hs00909240_m1 |
| ASCL1 | Hs00269932_m1 | GFAP | Hs00909238_g1 |
| ATOH1 | Hs00944192_s1 | GUSB | Hs00939627_m1 |
| CNP | Hs00263981_m1 | HOXA4 | Hs01573270_m1 |
| CXCL12 | Hs03676656_mH | HOXA5 | Hs00430330_m1 |
| CXCR4 | Hs00607978_s1 | HOXB6 | Hs00980016_m1 |
| DCX | Hs00167057_m1 | HOXB9 | Hs00256886_m1 |
| DLK1 | Hs00171584_m1 | HOXC6 | Hs00171690_m1 |
| DNMT3B | Hs00171876_m1 | HOXC9 | Hs00396786_m1 |
| EMX2 | Hs00244574_m1 | HOXD1 | Hs00707081_s1 |
| FOXA1 | Hs04187555_m1 | HPRT1 | Hs02800695_m1 |
| FOXG1 | Hs01850784_s1 | ITGA1 | Hs00235006_m1 |
| FOXO1 | Hs01054576_m1 | ITGB1 | Hs01127536_m1 |
| DLX2 | Hs00269993_m1 | HOXA5 | Hs00430330_m1 |
| NKX2-6 | Hs01045726_m1 | SIX3 | Hs00193667_m1 |
| KLF4 | HS00358836_M1 | PAX6 | Hs01088114_m1 |
| LEFTY1 | HS00764128_S1 | PDGFRA | Hs00998018_m1 |
| LEFTY2 | HS00745761_S1 | PGK1 | Hs00943178_g1 |
| MAP2 | HS00258900_M1 | POU5F1 | Hs00999632_g1 |
| MBP | HS00921945_M1 | PPIA | Hs04194521_s1 |
| MSI2 | HS00292670_M1 | RPLP0 | Hs99999902_m1 |
| MYC | HS00153408_M1 | S100B | Hs00902901_m1 |
| NANOG | HS04260366_G1 | SEMA3A | Hs00173810_m1 |
| NES | HS04187831_G1 | SLC1A3 | Hs00188193_m1 |
| NEUROG1 | HS01029249_S1 | SOX1 | Hs01057642_s1 |
| NFIA | HS00325656_M1 | SOX10 | Hs00366918_m1 |
| NFIB | HS01029174_M1 | SOX10 | Hs00199510_m1 |
| NKX2.1 | HS00968940_M1 | SOX2 | Hs01053049_s1 |
| NODAL | HS00415443_M1 | SOX2 | Hs01072517_s1 |
| NOTCH1 | HS01062014_M1 | STAT3 | Hs00374280_m1 |
| NOTCH2 | HS01050702_M1 | SYP | Hs00300531_m1 |
| OLIG1 | HS00744293_S1 | TBP | Hs00427620_m1 |
| OLIG2 | HS00300164_S1 | TDGF1 | Hs02339499_g1 |
| OTX2 | HS00222238_M1 | TUBB3 | Hs00801390_s1 |
| PAX6 | HS00240871_M1 | UBC | Hs01871556_s1 |
| PAX6 | HS01088114_M1 | DLK1 | Hs05635283_s1 |
| IRX3 | HS01124217_G1 | NKX2-1 | Hs00968940_m1 |

### Example 2: Efficient Direction of hiPSCs to a Cervical Spinal Cord NPC Fate

The first step of generating cerNPC is generation of unpatterned NPCs using established method. HiPSCs cultured under feeder-free conditions were differentiated into NPCs by switching the media from mTSR1 media to N2B27 media supplemented with dual SMAD inhibitors (LDN-19318, SB-431542) and CHIR-99021. qPCR analysis revealed a rapid loss of expression of the pluripotent markers POU5F1 (OCT4) and NANOG, whereas SOX2 expression was persistent. No expression of the neuroectoderm cell fate determinant PAX6 was noted at this step. Rosette forming cells were obtained 10 d after neural induction and unpatterned NPCs after 14 days. The resulting cells (unpatterned NPCs) shown to express fore brain (cortical) identity markers like FoxG1, Otx2 and Emx2.

The first step to determine the transcription factors (TFs) that can be used for patterning fbNPCs to cerNPC is based on systematic comparative genome-wide gene expression analyses between fbNPCs and cerNPC, to use TFs that are highly expressed in cerNPC compared to fbNPC. First, the expression signature of adult derived fbNPCs (cortical), cerNPCs and thoracic-NPCs was attempted to be identified.

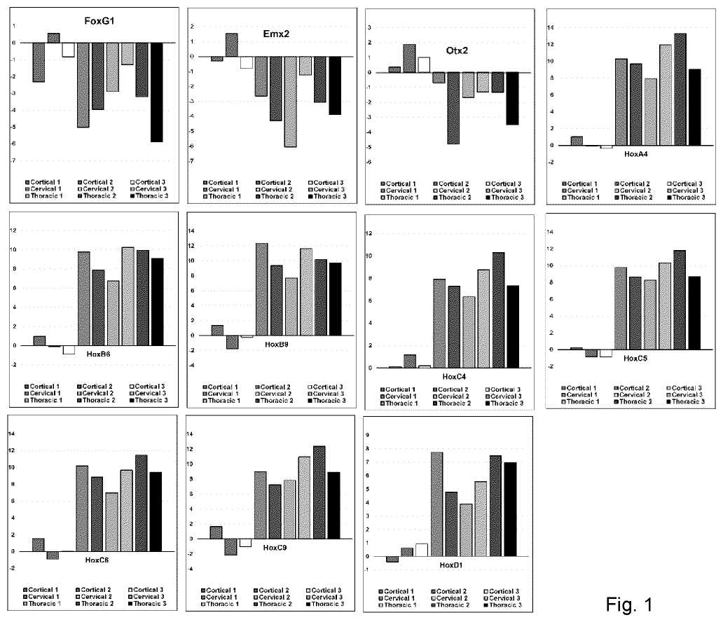

Gene expression profiling has demonstrated that FoxG1m Emx2, Otx2 and Gbx2 are the main markers of forebrain identity NPCs and the HoxB5, HoxC5 and Hox A6 are the main markers of cerNPCs (FIG. 1).

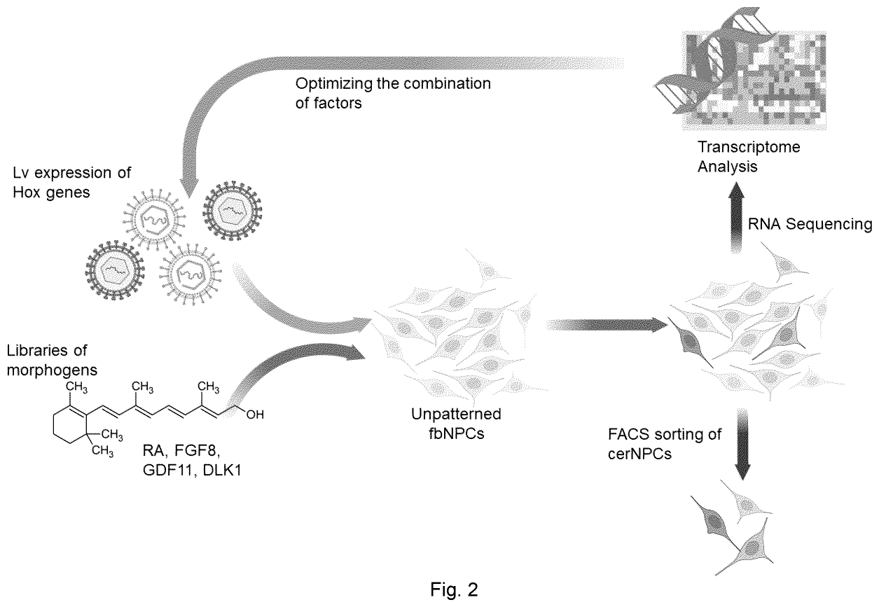

A forward genetics approach was then used to find the best approach to pattern fbNPCs towards cerNPCs. This approach is explained in FIG. 2. Using this approach, a screen was performed first to identify factors that can be used to pattern fbNPCs toward cerNPCs as much as possible, and based on gene expression analysis data, the patterning approach was refined with expression of Hox gene transcription factors.

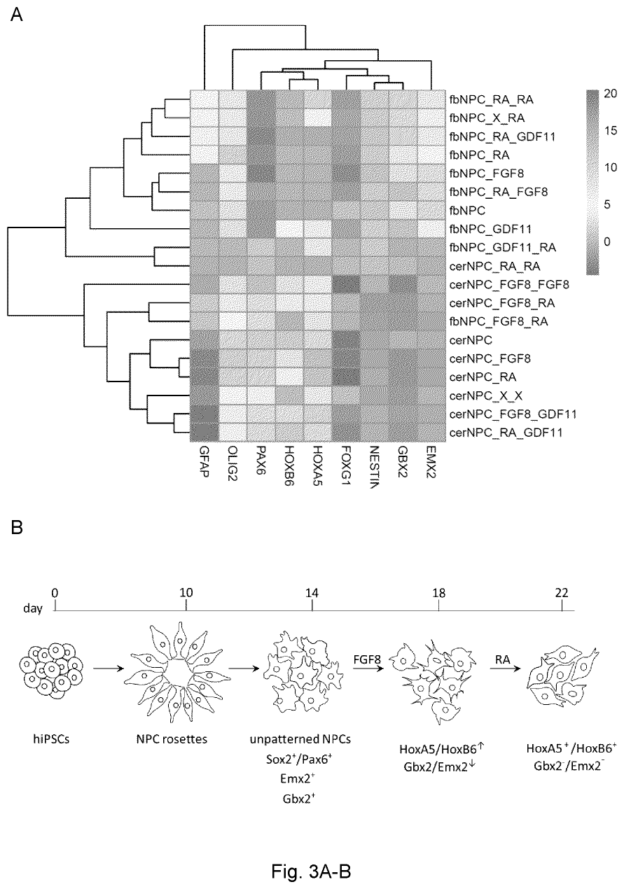

In a screen to find the best combination of morphogens or small molecules that can pattern fbNPCs towards cerNPCs, fbNPCs were treated with different factors (like GDF11, FGF8, Retinoic Acid) individually or combination for 4 days or 8 days. Based on gene expression profiling, overall treatment of fbNPCs with Retinoic Acid (RA) reduces EMX2, FoxG1 and GBx2. Also, Retinoic acid reduces Nestin expression but increases Pax6. Treatment of cerNPCs with RA reduces FoxG1, but increases the GBX and EMX2. Treatment with GDF11 did not show so much effect on changing the expression profile of the cells. So the best treatment to make fbNPCs as close to cerNPCs is 4 days with FGF8 and another 4 days with RA (FIG. 3).

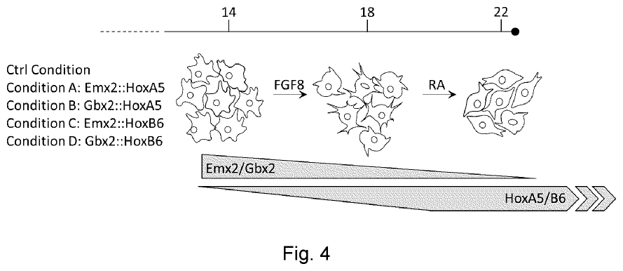

The cells that were generated using this method were not stable and after culturing cells back in the regular N2/B27 media the expression level of HoxA5/B6 started to decrease. Continued treatment with RA for longer period resulted in more canalization of cells towards thoracic or lumber identity. Therefore, method to stabilize the identity of NPCs in cervical fate was needed. The staged gene activation suggests that small-molecule-driven fbNPC to cerNPC patterning is a hierarchical transcriptional activation process, in which the efficient activation of cell-fate-determining genes by small molecules may initiate and stabilize an auto-regulatory loop of the identity-specific transcriptional program and may further stimulate the expression of downstream regulatory genes to establish cervical identity properties. The master transcription factors, HoxA5 and HoxB6 are considered the major determinants of cervical specific cell identities. Although these findings show that a small molecule cocktail (containing FGF8 and RA) is sufficient to activate the expression of cervical specific NPCs, but their effect was not stable. Therefore, it was decided to combine the FGF8 and RA paradigm with the findings from forward genetic approach to overexpress HoxB6 or A5 under Emx2 or Gbx2 promoters in combination with the FGF8/RA method (FIG. 4).

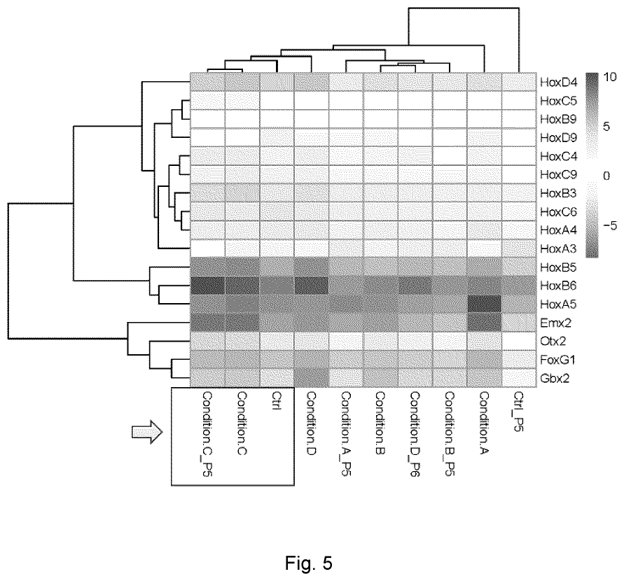

According to the gene expression analysis, condition C (which is expression of HoxB6 under Emx2) resulted in the most similar and most stable expression signature to cerNPCs (FIG. 5).

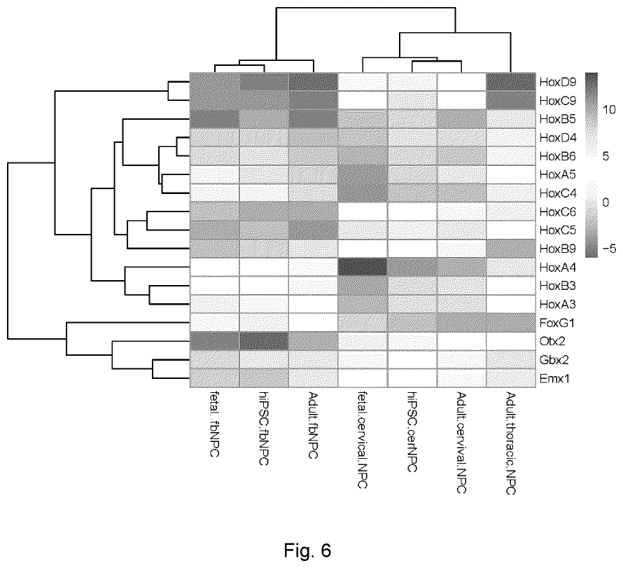

In the last step the gene expression signature of cells that were generated using condition C method were compared with the adult and fetal derived cervical NPCs, and their similarity was confirmed (FIG. 6).

### Example 3: cerNPCs are Tripotent In Vitro

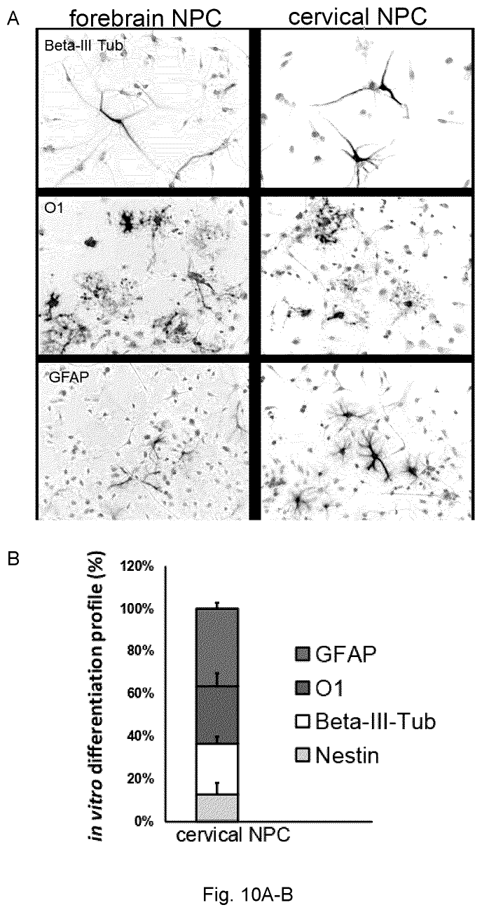

Next, developmental potential of cervical spinal NPCs was examined by assessing their capacity for differentiation into the three main neural lineages. To induce differentiation, NPCs were cultured in the absence of EGF and FGF but in the presence 0.1% FBS. Differentiated cells showed neuronal morphology and expressed the neuronal marker β-III-tubulin (FIG. 10). Differentiation to a neuronal fate was comparable with forebrain NPCs. Quantification of the number of GFAP positive cells, a marker for astrocytes and 01-positive cells, a marker of oligodendrocytes demonstrated the same differentiation profile between cervical and forebrain NPCs.

### Example 4: cerNPCs Survived, Migrated and Differentiated in the Injured Spinal Cord

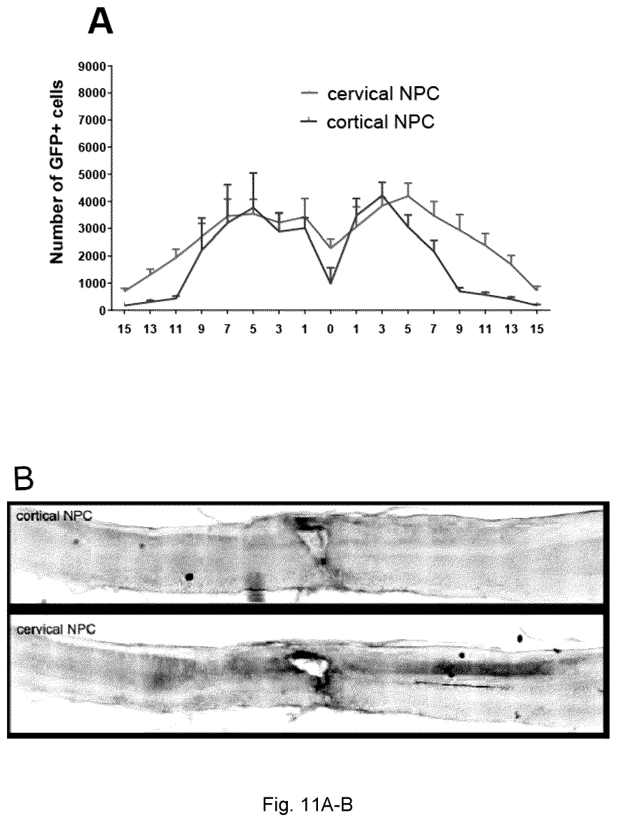

To investigate the in vivo behavior of cervical NPCs vs forebrain NPCs after transplantation, T-cell deficient RNU rats received a C6/7 cervical SCI followed by cell transplantation at 2 weeks post-injury. Transplanted cells (GFP+) were found in both the white and gray matter. Grafted cervical NPCs cells were located mainly in grey matter Rexed laminae I, II, VIII and IX and they showed a tendency to migrate along corticospinal and rubrospinal tracts (FIG. 11). They have migrated as far as 15 mm rostral and caudal from the epicenter and predominantly migrated along white matter tracts (FIG. 11).

Quantification of GFP+ cells revealed extensive transplanted cell survival within the injured spinal cord (FIG. 11). The cell survival rate was higher in the cervical NPCs group (37±24%) as compared to the forebrain-NPC group (29±65%).

### Example 5: In Vivo Differentiation Profile of Transplanted cerNPCs

Next, the in vivo differentiation profile of transplanted cervical and forebrain hiPSC-NPCs was assessed using immunohistochemistry (IHC). The majority of transplanted cells (GFP⁺ cells) in both groups differentiated to mature oligodendrocytes while few remained as immature oligodendrocytes. In fact, in several animals no detectable GFP⁺/Olig2⁺ cells could be found indicating that after 8 weeks most transplanted NPC-derived oligodendrocytes have matured. GFP colocalization with GFAP cells, was more frequently observed in the forebrain group than the GDNF-hiPSC-NPC transplanted rats, although were not significantly different. GFP colocalization with Fox3 (NeuN) equivalent in both groups. For cortical cells this Nestin positive population primarily expressed FoxG1 as opposed to the Nestin population of cervical NPCs. Conversely, in the Nestin negative population HoxA4 expression was barely detected in cortical NPCs but was in cervical NPCs.

### Example 6: Transplanted Cervical Spinal NPCs Differentiate to Spinal Neuronal Subtypes within the Injured Spinal Cord

The specific neuronal subtypes adopted by cervical spinal cord NPCs were further identified. Spinal cervical generated a variety of spinal interneuronal subtypes, including Chx10+ excitatory V2a interneurons, a type of propriospinal neuron, 3 months post-grafting. At 3 months post-grafting, cells also expressed FoxP1 (for excitatory neurons), choline acetyltransferase (ChAT; for cholinergic motor neurons and premotor interneurons), HB9 (for motor neurons), and Isl1 (for motor neurons). These are all typical markers of mature spinal cord neurons. Notably, these spinal cord cervical NPCs predominantly adopted excitatory neuronal fates.

### Example 7: Transplantation of cerNPCs Contributes to Functional Recovery from Spinal Cord Injury

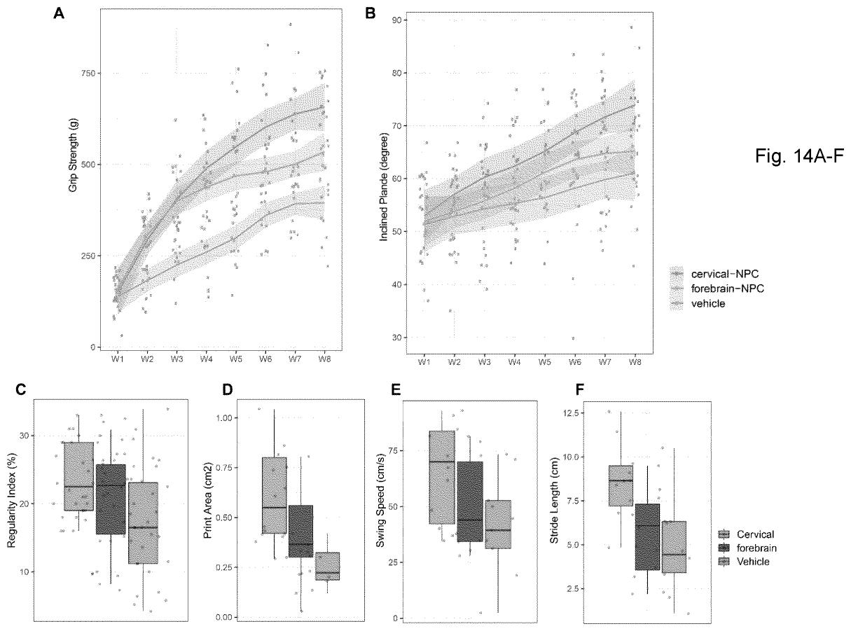

Next, it was determined whether transplantation of forebrain- or cervical-NPCs was associated with functional recovery. Forelimb strength and trunk stability were assessed with grip strength and inclined plane behavioral tasks¹⁰. All injured animals consistently recovered forelimb grip strength over the assessment period, although recovery trajectories diverged at approximately 4 weeks post-transplantation. There was a significant improvement in forelimb grip strength in both forbrain and cervical groups compared to the vehicle control group (p<0.05) (FIG. 14A). Although a trend toward better recovery was measured in both groups for the inclined plane test, the recovery was significantly better in cervical NPCs compared to forebrain-NPCs (FIG. 14B). Using the CatWalk (Noldus Inc.) digital gait analysis system, several static and dynamic parameters of locomotion relevant to cervical SCI were quantified at 8 weeks post transplantation. All injured groups exhibited abnormal walking patterns, slow rates of locomotion, and abnormal paw prints (FIG. 14C-F). Forelimb swing speed was significantly improved in rats transplanted with cervical NPCs versus the vehicle control but not for forebrain NPC (FIG. 14C-F). While the forelimb print area and stride length were significantly improved in cervical NPCs transplanted animals, they did not show improvement for forebrain cells. Furthermore, the regularity index, which is an indicator of coordination between all four limbs, was significantly improved for in cervical NPCs (FIG. 14C-F).

### Example 8: cerNPCs Show Better Electrical Conductance Over the Injury Site, Compared to fbNPCs

As cerNPCs produced more neurons, the overall number of exogenous-endogenous synaptic connections, particularly excitatory ones, is likely higher. These new connections could potentially contribute to greater electrical transmission across the injury site. To test this, electrically-evoked compound action potential (CAP) transmission across the injury site (C5-T1) was analyzed. The CAP amplitude was significantly higher in the cerNPC transplant group compared to control fbNPCs. This could reflect a lower number of neurons, and therefore fewer new synaptic connections in fbNPCs cells and compared to cerNPCs.

Although the disclosure has been described with reference to certain specific embodiments, various modifications thereof will be apparent to those of ordinary skill in the art. Any examples provided herein are included solely for the purpose of illustrating the disclosure and are not intended to limit the disclosure in any way. Any drawings provided herein are solely for the purpose of illustrating various aspects of the disclosure and are not intended to be drawn to scale or to limit the disclosure in any way. The scope of the claims appended hereto should not be limited by the preferred embodiments set forth in the above description, but should be given the broadest interpretation consistent with the present specification as a whole. The disclosures of all documents cited herein are incorporated herein by reference as if set forth in their entirety.

### CITED REFERENCES

1. Bonner, J. F. & Steward, O. Repair of spinal cord injury with neuronal relays: From fetal grafts to neural stem cells. Brain Res. 1619, 115-123 (2015).
2. Kumamaru, H. et al. Therapeutic activities of engrafted neural stem/precursor cells are not dormant in the chronically injured spinal cord. Stem Cells 31, 1535-47 (2013).
3. Tscherter, A., Heidemann, M., Kleinlogel, S. & Streit, J. Embryonic Cell Grafts in a Culture Model of Spinal Cord Lesion: Neuronal Relay Formation Is Essential for Functional Regeneration. Front. Cell. Neurosci. 10, (2016).
4. Karimi-Abdolrezaee, S., Eftekharpour, E., Wang, J., Morshead, C. M. & Fehlings, M. G. Delayed transplantation of adult neural precursor cells promotes remyelination and functional neurological recovery after spinal cord injury. J. Neurosci. Off. J. Soc. Neurosci. 26, 3377-3389 (2006).
5. Kadoya, K. et al. Spinal cord reconstitution with homologous neural grafts enables robust corticospinal regeneration. Nat. Med. (2016) doi: 10.1038/nm.4066.
6. Kirkeby, A. et al. Generation of Regionally Specified Neural Progenitors and Functional Neurons from Human Embryonic Stem Cells under Defined Conditions. Cell Rep. 1, 703-714 (2012).
7. Helms, A. W. & Johnson, J. E. Specification of dorsal spinal cord interneurons. Curr. Opin. Neurobiol. 13, 42-49 (2003).
8. Butts, J. C. et al. Differentiation of V2a interneurons from human pluripotent stem cells. Proc. Natl. Acad. Sci. U.S.A 114, 4969-4974 (2017).
9. Zholudeva, L. V. et al. Transplantation of Neural Progenitors and V2a Interneurons after Spinal Cord Injury. J. Neurotrauma 35, 2883-2903 (2018).
10. Wilcox, J. T. et al. Generating level-dependent models of cervical and thoracic spinal cord injury: Exploring the interplay of neuroanatomy, physiology, and function. Neurobiol. Dis. 105, 194-212 (2017).
11. Surmacz, B. et al. Directing differentiation of human embryonic stem cells toward anterior neural ectoderm using small molecules. Stem Cells Dayt. Ohio 30, 1875-1884 (2012).
12. Hitoshi, S., Tropepe, V., Ekker, M. & van der Kooy, D. Neural stem cell lineages are regionally specified, but not committed, within distinct compartments of the developing brain. Dev. Camb. Engl. 129, 233-244 (2002).
13. Fehlings, M. G. & Tator, C. H. The relationships among the severity of spinal cord injury, residual neurological function, axon counts, and counts of retrogradely labeled neurons after experimental spinal cord injury. Exp. Neurol. 132, 220-228 (1995).
14. Chambers, S. M. et al. Highly efficient neural conversion of human ES and iPS cells by dual inhibition of SMAD signaling. Nat. Biotechnol. 27, 275-280 (2009).
15. Wilcox, J. T., Satkunendrarajah, K., Zuccato, J. A., Nassiri, F. & Fehlings, M. G. Neural precursor cell transplantation enhances functional recovery and reduces astrogliosis in bilateral compressive/contusive cervical spinal cord injury. Stem Cells Transl. Med. 3, 1148-1159 (2014).
16. García-Alías, G., Barkhuysen, S., Buckle, M. & Fawcett, J. W. Chondroitinase ABC treatment opens a window of opportunity for task-specific rehabilitation. Nat. Neurosci. 12, 1145-1151 (2009).
17. Bresnahan, J. C., Beattie, M. S., Todd III, F. D. & Noyes, D. H. A behavioral and anatomical analysis of spinal cord injury produced by a feedback-controlled impaction device. Exp. Neurol. 95, 548-570 (1987).
18. Dai, H. et al. Delayed rehabilitation with task-specific therapies improves forelimb function after a cervical spinal cord injury. Restor. Neurol. Neurosci. 29, 91-103 (2011).
19. Miyagi, M. et al. Assessment of gait in a rat model of myofascial inflammation using the CatWalk system. Spine 36, 1760-1764 (2011).
20. Basso, D. M., Beattie, M. S. & Bresnahan, J. C. A sensitive and reliable locomotor rating scale for open field testing in rats. J. Neurotrauma 12, 1-21 (1995).
21. Tao, Y. & Zhang, S. C. Neural Subtype Specification from Human Pluripotent Stem Cells. Cell Stem Cell 19, 573-586 (2016).
22. Wen, Y. & Jin, S. Production of neural stem cells from human pluripotent stem cells. J. Biotechnol. 188, 122-129 (2014).
23. Salewski, R. P. et al. The generation of definitive neural stem cells from PiggyBac transposon-induced pluripotent stem cells can be enhanced by induction of the NOTCH signaling pathway. Stem Cells Dev. 22, 383-396 (2013).
24. Woo, S. M. et al. Notch signaling is required for maintaining stem-cell features of neuroprogenitor cells derived from human embryonic stem cells. BMC Neurosci. 10, 97 (2009).
25. Smukler, S. R., Runciman, S. B., Xu, S. & van der Kooy, D. Embryonic stem cells assume a primitive neural stem cell fate in the absence of extrinsic influences. J. Cell Biol. 172, 79-90 (2006).
26. Chambers, S. M. et al. Highly efficient neural conversion of human ES and iPS cells by dual inhibition of SMAD signaling. Nat. Biotechnol. 27, 275-280 (2009).
27. Lippmann, E. S. et al. Deterministic HOX patterning in human pluripotent stem cell-derived neuroectoderm. Stem Cell Rep. 4, 632-644 (2015).
28. Joyner, A. L., Liu, A. & Millet, S. Otx2, Gbx2 and Fgf8 interact to position and maintain a mid-hindbrain organizer. Curr. Opin. Cell Biol. 12, 736-741 (2000).
29. Liu, J. P., Laufer, E. & Jessell, T. M. Assigning the Positional Identity of Spinal Motor Neurons: Rostrocaudal Patterning of Hox-c Expression by FGFs, Gdf11, and Retinoids. Neuron 32, 997-1012 (2001).
30. Resende, T. P. et al. Sonic hedgehog in temporal control of somite formation. Proc. Natl. Acad. Sci. U.S.A 107, 12907-12912 (2010).
31. Aulehla, A. et al. Wnt3a plays a major role in the segmentation clock controlling somitogenesis. Dev. Cell 4, 395-406 (2003).
32. Delfino-Machín, M., Lunn, J. S., Breitkreuz, D. N., Akai, J. & Storey, K. G. Specification and maintenance of the spinal cord stem zone. Dev. Camb. Engl. 132, 4273-4283 (2005).
33. Olivera-Martinez, I., Harada, H., Halley, P. A. & Storey, K. G. Loss of FGF-dependent mesoderm identity and rise of endogenous retinoid signalling determine cessation of body axis elongation. PLoS Biol. 10, e1001415 (2012).
34. Okada, Y., Shimazaki, T., Sobue, G. & Okano, H. Retinoic-acid-concentration-dependent acquisition of neural cell identity during in vitro differentiation of mouse embryonic stem cells. Dev. Biol. 275, 124-142 (2004).
35. Irioka, T., Watanabe, K., Mizusawa, H., Mizuseki, K. & Sasai, Y. Distinct effects of caudalizing factors on regional specification of embryonic stem cell-derived neural precursors. Dev. Brain Res. 154, 63-70 (2005).
36. Nordström, U., Jessell, T. M. & Edlund, T. Progressive induction of caudal neural character by graded Wnt signaling. Nat. Neurosci. 5, 525-532 (2002).
37. Derossi, D., Williams, E. J., Green, P. J., Dunican, D. J. & Doherty, P. Stimulation of mitogenesis by a cell-permeable PI 3-kinase binding peptide. Biochem. Biophys. Res. Commun. 251, 148-152 (1998).

We claim:

1. An engineered cervical spinal cord-specific neural progenitor cell (cerNPC) having an increased expression of one or more Hox genes and a decreased expression of one or more of Gbx2, Otx2 and FoxG1 relative to a non-specific neural progenitor cell (NPC) or a forebrain-specific neural progenitor cell (fbNPC).

2. The engineered cerNPC of claim 1, wherein the cerNPC is capable of differentiating into neurons, astrocytes and/or oligodendrocytes.

3. The engineered cerNPC of claim 1 or 2, wherein the cerNPC is derived from a non-specific NPC that expresses one or more detectable markers that are Sox2, Pax6, nestin and/or vimentin.

4. The engineered cerNPC of any one of claims 1-3, wherein the one or more Hox genes comprise one or more of HoxA3, HoxA4, HoxA5, HoxB3, HoxB5, HoxB6, HoxB9, HoxC4, HoxC5, HoxC6, HoxC9, HoxD4 and HoxD9.

5. The engineered cerNPC of any one of claims 1-4, wherein the one or more Hox genes comprise HoxA5 and/or HoxB6.

6. The engineered cerNPC of any one of claims 1-5, wherein the one or more Hox genes are expressed from an Emx2 promoter, a FoxG1 promoter, a Gbx2 promoter and/or an Otx2 promoter.

7. The engineered cerNPC of any one of claims 1-6, wherein the one or more Hox genes are expressed from an Emx2 promoter.

8. The engineered cerNPC of any one of claims 1-5, wherein the one or more Hox genes are expressed from an inducible promoter.

9. The engineered cerNPC of any one of claims 1-8, wherein the cerNPC is a human cerNPC.

10. The engineered cerNPC of any one of claims 3-9, wherein the non-specific NPC that expresses the one or more detectable markers that are Sox2, Pax6, nestin and/or vimentin is (a) derived from a stem or progenitor cell, or (b) reprogrammed from a differentiated cell.

11. The engineered cerNPC of claim 10, wherein the stem or progenitor cell is derived from adult tissue, fetal tissue, or embryonic stem cells.

12. The engineered cerNPC of claim 10 or 11, wherein the stem or progenitor cell is an induced pluripotent stem cell.

13. The engineered cerNPC of any one of claims 1-12, wherein synaptic connectivity of the cerNPC with endogenous neurons is increased relative to synaptic connectivity of the NPC or the fbNPCs with endogenous neurons.

14. The engineered cerNPC of any one of claims 1-13, wherein electrical conductance of the cerNPC across a site of spinal cord injury is increased relative to electrical conductance of the NPC or the fbNPCs across a site of spinal cord injury.

15. A pharmaceutical composition comprising the engineered cerNPC of any one of claims 1-14 and a pharmaceutically acceptable carrier, diluent or excipient.

16. The pharmaceutical composition of claim 15, wherein the pharmaceutically acceptable carrier is a xenogen-free culture medium or matrix.

17. The pharmaceutical composition of claim 15, wherein the pharmaceutically acceptable carrier is cerebrospinal fluid or synthetic cerebrospinal fluid.

18. A method of treating a neurological disease or disorder in a subject, the method comprising administering a therapeutically effective amount of the engineered cerNPC of any one of claims 1-14 or the pharmaceutical composition of any one of claims 15-17 to the subject.

19. The method of claim 18, wherein the administering comprises transplanting the engineered cerNPC to the brain or spinal cord of the subject.

20. The method of claim 18 or 19, wherein the neurological disease or disorder is Parkinson's disease, Alzheimer's disease, Huntington's disease, Amyotrophic lateral sclerosis, Friedreich's ataxia, Lewy body disease, spinal muscular atrophy, multiple system atrophy, dementia, schizophrenia, paralysis, multiple sclerosis, spinal cord injury, brain injury, stroke, cranial nerve disorders, peripheral sensory neuropathies, epilepsy, prion disorders, Creutzfeldt-Jakob disease, Alper's disease, cerebellar/spinocerebellar degeneration, Batten disease, corticobasal degeneration, Bell's palsy, Guillain-Barre syndrome, Pick's disease, or autism.

21. The method of any one of claims 18-20, wherein the neurological disease or disorder is spinal cord injury.

22. Use of the engineered cerNPC of any one of claims 1-14 or the pharmaceutical composition of any one of claims 15-17 in the manufacture of a medicament for treating a neurological disease or disorder.

23. The use of claim 22, wherein the neurological disease or disorder is Parkinson's disease, Alzheimer's disease, Huntington's disease, Amyotrophic lateral sclerosis, Friedreich's ataxia, Lewy body disease, spinal muscular atrophy, multiple system atrophy, dementia, schizophrenia, paralysis, multiple sclerosis, spinal cord injury, brain injury, stroke, cranial nerve disorders, peripheral sensory neuropathies, epilepsy, prion disorders, Creutzfeldt-Jakob disease, Alper's disease, cerebellar/spinocerebellar degeneration, Batten disease, corticobasal degeneration, Bell's palsy, Guillain-Barre syndrome, Pick's disease, or autism.

24. The use of claim 22 or 23, wherein the neurological disease or disorder is spinal cord injury.

25. A method of producing cervical spinal cord-specific neural progenitor cells (cerNPCs), the method comprising:
(a) expressing one or more Hox genes in stem or progenitor cells;
(b) differentiating the stem or progenitor cells into non-specific neural progenitor cells (NPCs);
(c) culturing the non-specific NPCs for about 4 days in media supplemented with B27, N2, about 30-50 ng/ml of FGF2 or an agonist or synthetic analog thereof, and about 150-250 ng/ml of FGF8b or an agonist or synthetic analog thereof to generate posteriorized NPCs;
(d) culturing the posteriorized NPCs for about 4 days in media supplemented with B27, N2, about 0.075-0.125 UM of retinoic acid (RA) or an agonist or synthetic analog thereof, and optionally about 75-125 μg/ml of Wnt3a or an agonist or synthetic analog thereof to generate caudalized NPCs; and
(e) culturing the caudalized NPCs in media supplemented with B27, N2, about 7.5-12.5 ng/ml of FGF2 or an agonist or synthetic analog thereof, about 7.5-12.5 ng/ml of EGF or an agonist or synthetic analog thereof and about 0.75-1.25 UM of 740Y-P or an agonist or synthetic analog thereof to generate cerNPCs.

26. The method of claim 25, wherein the one or more Hox genes comprise one or more of HoxA3, HoxA4, HoxA5, HoxB3, HoxB5, HoxB6, HoxB9, HoxC4, HoxC5, HoxC6, HoxC9, HoxD4 and HoxD9.

27. The method of claim 25 or 26, wherein the one or more Hox genes comprise HoxA5 and/or HoxB6.

28. The method of any one of claims 25-27, wherein the one or more Hox genes are expressed from an Emx2 promoter, a FoxG1 promoter, a Gbx2 promoter and/or an Otx2 promoter.

29. The method of any one of claims 25-28, wherein the one or more Hox genes are expressed from an Emx2 promoter.

30. The method of any one of claims 25-27, wherein the one or more Hox genes are expressed from an inducible promoter.

31. The method of any one of claims 25-30, wherein the stem or progenitor cells are human cells.

32. The method of any one of claims 25-31, wherein the stem or progenitor cells are derived from adult tissue, fetal tissue, or embryonic stem cells.

33. The method of any one of claims 25-32, wherein the stem or progenitor cells are induced pluripotent stem cells.

34. The method of any one of claims 25-33, wherein the non-specific NPCs express one or more detectable markers that are Sox2, Pax6, nestin and/or vimentin.

35. The method of any one of claims 25-34, wherein step (b) comprises differentiating the stem or progenitor cells into the non-specific NPCs using embryoid body formation or dual SMAD inhibition.

36. The method of any one of claims 25-35, wherein step (e) comprises culturing the caudalized NPCs for a period of about 3-10 passages to generate the cerNPCs.

37. The method of any one of claims 25-36, wherein:
in step (c), the FGF2 or the agonist or synthetic analog thereof is present at a concentration of about 40 ng/ml and the FGF8b or the agonist or synthetic analog thereof is present at a concentration of about 200 ng/ml; and/or
in step (d), the RA or the agonist or synthetic analog thereof is present at a concentration of about 0.1 μM and the Wnt3a or the agonist or synthetic analog thereof is present at a concentration of about 100 μg/ml; and/or
in step (e), the FGF2 or the agonist or synthetic analog thereof is present at a concentration of about 10 ng/ml, the EGF or the agonist or synthetic analog thereof is present at a concentration of about 10 ng/ml, and the 740Y-P or the agonist or synthetic analog thereof is present at a concentration of about 1 μM.

38. The method of any one of claims 25-37, wherein the synthetic analog of RA is EC23.

39. The method of any one of claims 25-38, wherein the culturing is performed on a substrate or matrix comprising poly-L-lysine, laminin, poly-L-lysine/laminin, fibronectin, vitronectin, collagen, Matrigel™, or Geltrex™.

40. The method of claim 39, wherein the culturing is performed in plates coated with poly-L-lysine/laminin.

41. The method of any one of claims 25-40, wherein the cerNPCs are capable of differentiating into neurons, astrocytes and/or oligodendrocytes.

42. The method of any one of claims 25-41, wherein the cerNPCs have an increased expression of one or more Hox genes and a decreased expression of one or more of Gbx2, Otx2 and FoxG1 relative to the non-specific NPC or a forebrain-specific neural progenitor cell (fbNPC).

43. The method of any one of claims 25-42, further comprising a step of formulating the cerNPCs with a pharmaceutically acceptable carrier, diluent or excipient.

44. The method of claim 43, wherein the pharmaceutically acceptable carrier is a xenogen-free culture medium or matrix.

45. The method of claim 43, wherein the pharmaceutically acceptable carrier is cerebrospinal fluid or synthetic cerebrospinal fluid.
